SEQUENCE LISTING (5 sequences)

SEQ ID NO: 1 - DNA, 30 bp, synthetic construct
  source 1..30: mol_type = other DNA
cagtcgacgtccgtatagcctaagttcagt

SEQ ID NO: 2 - DNA, 83 bp, synthetic construct
  source 1..83: mol_type = other DNA
  misc_difference 15..21: note = n is any nucleotide other than C
  misc_difference 23..28: note = n is any nucleotide other than C
  misc_difference 31..35: note = n is any nucleotide other than C
  misc_difference 38..45: note = n is any nucleotide other than C
  misc_difference 48..53: note = n is any nucleotide other than C
  misc_difference 56..64: note = n is any nucleotide other than C
  misc_difference 66..72: note = n is any nucleotide other than C
  misc_difference 75..83: note = n is any nucleotide other than C
tagcggtcatggtannnnnnncnnnnnncgnnnnncgnnnnnnnntgnnnnnncgnnnnnnnnncnnnnnnntgnnnnnnnnn

SEQ ID NO: 3 - DNA, 83 bp, synthetic construct
  source 1..83: mol_type = other DNA
  misc_difference 15..21: note = n is any nucleotide other than C
  misc_difference 23..28: note = n is any nucleotide other than C
  misc_difference 31..35: note = n is any nucleotide other than C
  misc_difference 38..45: note = n is any nucleotide other than C
  misc_difference 48..53: note = n is any nucleotide other than C
  misc_difference 56..64: note = n is any nucleotide other than C
  misc_difference 66..72: note = n is any nucleotide other than C
  misc_difference 75..83: note = n is any nucleotide other than C
tagcggtcatggtannnnnnncnnnnnntgnnnnntgnnnnnnnntgnnnnnntgnnnnnnnnncnnnnnnntgnnnnnnnnn

SEQ ID NO: 4 - DNA, 83 bp, synthetic construct
  source 1..83: mol_type = other DNA
  misc_difference 15..21: note = n is any nucleotide other than C
  misc_difference 23..28: note = n is any nucleotide other than C
  misc_difference 31..35: note = n is any nucleotide other than C
  misc_difference 38..45: note = n is any nucleotide other than C
  misc_difference 48..53: note = n is any nucleotide other than C
  misc_difference 56..64: note = n is any nucleotide other than C
  misc_difference 66..72: note = n is any nucleotide other than C
  misc_difference 75..83: note = n is any nucleotide other than C
tagcggtcatggtannnnnnncnnnnnncgnnnnncgnnnnnnnncgnnnnnncgnnnnnnnnncnnnnnnncgnnnnnnnnn

SEQ ID NO: 5 - DNA, 82 bp, synthetic construct
  source 1..82: mol_type = other DNA
  misc_difference 15..21: note = n is any nucleotide other than C
  misc_difference 23..28: note = n is any nucleotide other than C
  misc_difference 31..35: note = n is any nucleotide other than C
  misc_difference 38..45: note = n is any nucleotide other than C
  misc_difference 48..53: note = n is any nucleotide other than C
  misc_difference 56..64: note = n is any nucleotide other than C
  misc_difference 66..72: note = n is any nucleotide other than C
  misc_difference 75..82: note = n is any nucleotide other than C
tagcggtcatggcannnnnnncnnnnnncgnnnnncgnnnnnnnncgnnnnnncgnnnnnnnnncnnnnnnncgnnnnnnnn

(19) United States
(12) Patent Application Publication
(10) Pub. No.: US 2024/0368686 A1
(43) Pub. Date: Nov. 7, 2024

(54) METHYLATION SEQUENCING METHODS AND COMPOSITIONS

(71) Applicant: ULTIMA GENOMICS, INC., Fremont, CA (US)

(72) Inventors: Zohar SHIPONY, Rehovot (IL); Florian OBERSTRASS, Redwood City, CA (US); Doron LIPSON, Ramat Hasharon (IL); Eti MEIRI, Shoham (IL); Omer BARAD, Mazkeret Batya (IL)

(21) Appl. No.: 18/657,167

(22) Filed: May 7, 2024

Related U.S. Application Data
(63) Continuation of application No. PCT/US2022/079395, filed on Nov. 7, 2022.
(60) Provisional application No. 63/263,743, filed on Nov. 8, 2021.
(60) Provisional application No. 63/306,977, filed on Feb. 4, 2022.

(51) Int. Cl. C12Q 1/6874; C12Q 1/6876

(52) U.S. Cl. CPC C12Q 1/6874; C12Q 1/6876; C12Q 2600/154

(57) ABSTRACT

Described herein is a composition, comprising: a first strand comprising a first portion and a first copy portion, wherein the first copy portion is a copy of the first portion except that substantially all cytosine bases in the first copy portion are methylated; and a second strand comprising a second portion and a second copy portion, wherein the second copy portion is a copy of the second portion except that substantially all cytosine bases in the second copy portion are methylated. In some implementations, at least one cytosine base in the first portion or the second portion is not methylated.

### CROSS-REFERENCE TO RELATED APPLICATIONS

This application is a continuation of International Patent Application No. PCT/US2022/079395, filed on Nov. 7, 2022, which claims the priority benefit of U.S. Provisional Patent Application No. 63/263,743, filed on Nov. 8, 2021; and U.S. Provisional Patent Application No. 63/306,977, filed on Feb. 4, 2022; the contents of each of which are incorporated herein by reference in its entirety.

### REFERENCE TO AN ELECTRONIC SEQUENCE LISTING

The contents of the electronic sequence listing (165272001940SEQLIST.xml; Size: 18,537 bytes; and Date of Creation: Nov. 4, 2022) is herein incorporated by reference in its entirety.

### FIELD OF THE INVENTION

Described herein are methods of sequencing a polynucleotide, including methods for determining a methylation profile for the polynucleotide.

### BACKGROUND

Next-generation sequencing (NGS) methods allow for high throughput sequencing of polynucleotides, giving insight into genetic profiles of patients and cancers. Methylation patterns on certain genes can be associated with certain aspects of a cancer, for example responsiveness to certain therapies or cancer driving mechanisms. However, NGS sequencing alone does not provide a methylation profile.

Chemical and enzymatic processes can selectively modify methylated or non-methylated cytosine bases. For example, treating a 5-methylated cytosine (5mC) with bisulfate can convert the methylated cytosine to a uracil base. This selective conversion can be used to identify methylated cytosine nucleotides in a target sequence. However, such a modification disrupts the nucleotide sequence, making it challenging to map a location of a methylated cytosine to a particular locus within the subject genome.

### BRIEF SUMMARY OF THE INVENTION

Described herein is a composition, comprising: a first strand comprising a first portion and a first copy portion, wherein the first copy portion is a copy of the first portion except that substantially all cytosine bases in the first copy portion are methylated; and a second strand comprising a second portion and a second copy portion, wherein the second copy portion is a copy of the second portion except that substantially all cytosine bases in the second copy portion are methylated. In some implementations, at least one cytosine base in the first portion or the second portion is not methylated.

Also described is a composition, comprising: a first strand comprising a first portion and a first copy portion, wherein the first copy portion is a copy of the first portion except that substantially all cytosine bases in the first copy portion are methylated cytosine, and substantially all bases in the first portion that correspond to cytosine bases in the first copy portion are methylated cytosine, uracil, or thymine; and a second strand comprising a second portion and a second copy portion, wherein the second copy portion is a copy of the second portion except that substantially all cytosine bases in the second copy portion are methylated cytosine, and substantially all bases in the second portion that correspond to cytosine bases in the second copy portion are methylated cytosine, uracil, or thymine.

Further described herein is a composition, comprising: a first strand comprising a first portion and a first copy portion, wherein the first copy portion is a copy of the first portion except that at least a portion of bases in the first portion that correspond to cytosine bases in the first copy portion are uracil or thymine; and a second strand comprising a second portion and a second copy portion, wherein the second copy portion is a copy of the second portion except that at least a portion of bases in the second portion that correspond to cytosine bases in the second copy portion are uracil or thymine. In some implementations, at least one cytosine base in the first portion or the second portion is not methylated.

In some implementations of any of the above constructs, the first strand and the second strand hybridize to each other in water at 25° C. In some implementations, the first strand is a reverse complement of the second strand. In some implementations, the first strand is substantially a reverse complement of the second strand (e.g., the first strand differs from the reverse complement of the second strand at one, two, three, four, or five loci).

In some implementations of any of the above constructs, the first copy portion is a reverse complement of the second copy portion.

In some implementations of any of the above constructs, the first portion and the first copy portion are separated by a first nucleic acid linker, and the second portion and the second copy portion are separated by a second nucleic acid linker. In some implementations, the first nucleic acid linker is a reverse complement of the second nucleic acid linker. In some implementations, the first nucleic acid linker or the second nucleic acid linker comprises a unique molecular identifier. In some implementations, the first nucleic acid linker or the second nucleic acid linker comprises a sample barcode. In some implementations, the first nucleic acid linker or the second nucleic acid linker is about 30 bases in length to about the length the first portion or the second portion. In some implementations, the first nucleic acid linker or the second nucleic acid linker is between about 20% and about 100% of a length of the first portion or the second portion.

In some implementations of any of the above constructs, the first strand comprises a first sequencing adapter sequence and the second strand comprises a second sequencing adapter sequence. The first sequencing adapter sequence and the second sequencing adapter sequence may comprise the same nucleic acid sequence. The first sequencing adapter sequence or the second sequencing adapter sequence can comprise a unique molecular identifier. The first sequencing adapter sequence or the second sequencing adapter sequence can comprise a sample barcode.

Further described herein is a method, comprising: performing extension reactions, in the presence of methylated cytosine, on a partially circular nucleic acid molecule comprising a first strand comprising a first template sequence and a second strand comprising a second template sequence, wherein the first template sequence is a reverse complement of the second template sequence, thereby generating a nucleic acid molecule comprising: a first strand comprising the first template sequence and a first copy portion, wherein the first copy portion is a copy of the first template sequence except that substantially all cytosine bases in the first copy portion are methylated; and a second strand comprising the second template sequence and a second copy portion, wherein the second copy portion is a copy of the second template sequence except that substantially all cytosine bases in the second copy portion are methylated. In some implementations, substantially all cytosine bases present in the extension reactions are methylated cytosine. In some implementations, the first template sequence or the second template sequence comprises at least one non-methylated cytosine.

Also described herein is a method, comprising: (a) providing: a template nucleic acid molecule comprising a first strand comprising a first template sequence and a second strand comprising a second template sequence, wherein the first template sequence is a reverse complement of the second template sequence; and an oligonucleotide set, comprising a first oligonucleotide, a second oligonucleotide, a third oligonucleotide, and a fourth oligonucleotide, wherein: a 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide; (b) ligating: a 3′ terminus of the first oligonucleotide to a 5′ terminus of the first strand, a 5′ terminus of the second oligonucleotide to a 3′ terminus of the second strand, a 5′ terminus of the third oligonucleotide to a 3′ terminus of the first strand, and a 3′ terminus of the fourth oligonucleotide to a 5′ terminus of the second strand; and (c) performing extension reactions in the presence of a nucleotide reagent comprising methylated cytosine bases, comprising: extending the 3′ terminus of the second oligonucleotide using, in order, a portion of the third oligonucleotide, the first strand and the first oligonucleotide as a template, extending the 3′ terminus of the third oligonucleotide using, in order, a portion of the second oligonucleotide, the second strand, and the fourth oligonucleotide as a template. In some implementations, substantially all cytosine bases in the nucleotide reagent are methylated cytosine bases.

In some implementations of the above method, the method further comprises crosslinking the second oligonucleotide to the third oligonucleotide. In some implementations, the crosslinker is a reversible crosslinker. In some implementations, the second oligonucleotide is crosslinked to the third oligonucleotide before the ligating. In some implementations, the second oligonucleotide is crosslinked to the third oligonucleotide after the ligating.

The above method can generate a composition comprising: a first construct strand comprising the first template sequence and a first copy portion, wherein the first copy portion is a copy of the first template sequence except that substantially all cytosine bases in the first copy portion are methylated; and a second construct strand comprising the second template sequence and a second copy portion, wherein the second copy portion is a copy of the second template sequence except that substantially all cytosine bases in the second copy portion are methylated. In some implementations, the first template sequence and the first copy portion are separated by a first nucleic acid linker, and the second template sequence and the second copy portion are separated by a second nucleic acid linker. In some implementations, the first nucleic acid linker is a reverse complement of the second nucleic acid linker. In some implementations, the first nucleic acid linker or the second nucleic acid linker comprises a unique molecular identifier. In some implementations, the first nucleic acid linker or the second nucleic acid linker comprises a sample barcode. In some implementations, the first nucleic acid linker or the second nucleic acid linker is about 30 bases in length to about the length the first template sequence or the second template sequence. In some implementations, the first nucleic acid linker or the second nucleic acid linker is between about 20% and about 100% of a length of the first template sequence or the second template sequence. In some implementations, the first nucleic acid linker and the second nucleic acid linker each have a known sequence.

In some implementations of the above method, the first construct strand comprises a first sequencing adapter sequence and the second construct strand comprises a second sequencing adapter sequence. In some implementations, the first sequencing adapter sequence and the second sequencing adapter sequence comprise the same nucleic acid sequence. In some implementations, the first sequencing adapter sequence or the second sequencing adapter sequence comprises a unique molecular identifier. In some implementations, the first sequencing adapter sequence or the second sequencing adapter sequence comprises a sample barcode.

In some implementations of the above method, the method further comprising converting non-methylated cytosine in the first construct strand or the second construct strand to uracil to generate a converted nucleic acid molecule comprising a first converted strand comprising a first converted template portion and a first converted copy portion, or a second converted strand comprising a second converted template portion and a second converted copy portion. Alternatively, the method comprises converting methylated cytosine in the first construct strand or the second construct strand to uracil to generate a converted nucleic acid molecule comprising a first converted strand comprising a first converted template portion and a first converted copy portion, or a second converted strand comprising a second converted template portion and a second converted copy portion.

In some implementations of the above method, the method further comprises amplifying the converted nucleic acid molecule, wherein uracil in the converted nucleic acid molecule is replaced with thymine.

In some implementations of the above method, the method further comprises generating first methylation profiling data for the first converted strand, the first methylation profiling data comprising: first sequencing data corresponding to the first copy portion indicating a nucleic acid sequence of the first template sequence; and second sequencing data corresponding to the first portion, wherein one or more differences between the first sequencing data and the second sequencing data are indicative of methylation status in the first template sequence. In some implementations, the first sequencing data and the second sequencing data of the first methylation profiling data are obtained from a same first strand sequencing read. In some implementations, generating second methylation profiling data for the second strand of the converted nucleic acid molecule, the second methylation profiling data comprising: third sequencing data corresponding to the second copy portion indicating a nucleic acid sequence of the second template sequence; and fourth sequencing data corresponding to the second portion, wherein one or more differences between the third sequencing data and the fourth sequencing data are indicative of methylation status in the second template sequence. In some implementations, the third sequencing data and the fourth sequencing data of the second methylation profiling data are obtained from a same second strand sequencing read. In some implementations, the first methylation profiling data or the second methylation profiling data comprises a location of methylated cytosine or non-methylated cytosine in the nucleic acid sequence of the first template sequence or the second template sequence. In some implementations, the first methylation profiling data or the second methylation profiling data comprises a density or signal intensity of methylated cytosine or non-methylated cytosine in the first template sequence or the second template sequence.

Methylation profiling data for the first converted strand or the second methylation profiling data for the second converted strand may be generated using a method that includes: hybridizing a sequencing primer to the first converted strand or the second converted strand to form a hybridized template; and generating sequencing data from the first converted copy portion or the second converted copy portion, comprising extending the sequencing primer by, for each of a plurality of sequencing flow steps, (i) providing, to the hybridized template, labeled nucleotides of a single base type, and (ii) detecting a signal indicating incorporation of a labeled nucleotide into the extending sequencing primer; and generating the methylation status data from the first converted template portion or the second converted template portion, comprising, extending the sequencing primer by, iteratively, (i) providing, to the hybridized template, a mixture of thymine, cytosine, and adenine nucleotides, (ii) providing, to the hybridized template, a mixture of cytosine and guanine bases, wherein at least a portion of the cytosine bases are labeled, and (iii) detecting a signal indicating incorporation of a labeled cytosine base into the extending sequencing primer. In some implementations, the method further comprises extending the sequencing primer through the nucleic acid linker between the generating the sequencing data and the generating the methylation status data. In some implementations, the method comprises extending the sequencing primer through the nucleic acid linker comprises for each of a plurality of extension flow steps, providing, to the hybridized template, a mixture of two or three different base types, wherein the two or three different base types provided to the hybridized template are selected based on a known sequence of the nucleic acid linker.

Also described herein is a method, comprising: converting, in a nucleic acid molecule, (i) non-methylated cytosine to uracil, or (ii) methylated cytosine to uracil, thereby generating a converted nucleic acid molecule; amplifying the converted nucleic acid molecule, thereby converting the uracil to thymine, to generate amplified converted nucleic acid molecules; hybridizing primers to the amplified nucleic acid molecules to form hybridized templates; and generating the methylation status data for at least a portion of the nucleic acid molecule, comprising extending the primers by, iteratively: (i) providing, to the hybridized templates, a mixture of thymine, cytosine, and adenine nucleotides, (ii) providing, to the hybridized templates, a mixture of cytosine and guanine bases, wherein at least a portion of the cytosine bases are labeled, and (iii) detecting a signal indicating incorporation of a labeled cytosine base into the extending sequencing primer. In some implementations, the method further comprises generating sequencing data for a second portion of the nucleic acid molecule, comprising, extending the primers by for each of a plurality of sequencing flow steps: (i) providing, to the hybridized templates, labeled nucleotides of a single base type, and (ii) detecting a signal indicating incorporation of a labeled nucleotide into the extending sequencing primer. In some implementations, the sequencing data is generated prior to generating the methylation status data. In some implementations, the method further comprises identifying a genomic locus for the methylation status data. In some implementations, identifying the genomic locus of the methylation status data comprises mapping the sequencing data to a reference sequence.

Further described herein is a method, comprising: (a) providing: a template nucleic acid molecule comprising a first strand comprising a first template sequence and a second strand comprising a second template sequence, wherein the first template sequence is a reverse complement of the second template sequence; and an oligonucleotide set, comprising a first oligonucleotide, a second oligonucleotide, a third oligonucleotide, and a fourth oligonucleotide, wherein: a 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide; (b) ligating: a 3′ terminus of the first oligonucleotide to a 5′ terminus of the first strand, a 5′ terminus of the second oligonucleotide to a 3′ terminus of the second strand, a 5′ terminus of the third oligonucleotide to a 3′ terminus of the first strand, and a 3′ terminus of the fourth oligonucleotide to a 5′ terminus of the second strand; (c) performing extension reactions in the presence of a nucleotide reagent comprising methylated cytosine bases, comprising: extending the 3′ terminus of the second oligonucleotide using, in order, a portion of the third oligonucleotide, the first strand and the first oligonucleotide as a template, extending the 3′ terminus of the third oligonucleotide using, in order, a portion of the second oligonucleotide, the second strand, and the fourth oligonucleotide as a template, thereby generating a nucleic acid molecule comprising: a first strand comprising the first template sequence portion and a first copy portion, and a second strand comprising the second template sequence portion and a second copy portion; (d) sequencing the first strand, comprising: hybridizing a sequencing primer to the first strand to form a hybridized template; generating first sequencing data for the first copy portion, comprising extending the sequencing primer by, for each of a plurality of sequencing flow steps according to a first flow order, (i) providing, to the hybridized template, labeled nucleotides of a single base type, and (ii) detecting a signal indicating incorporation of a labeled nucleotide into the extending sequencing primer; and generating second sequencing data for the first template sequence portion, comprising extending the sequencing primer by, for each of a plurality of sequencing flow steps according to a second flow order, (i) providing, to the hybridized template, labeled nucleotides of a single base type, and (ii) detecting a signal indicating incorporation of a labeled nucleotide into the extending sequencing primer, wherein the first flow order and the second flow order are different.

Also described herein is a method, comprising: (a) providing: a template nucleic acid molecule comprising a first strand comprising a first template sequence and a second strand comprising a second template sequence, wherein the first template sequence is a reverse complement of the second template sequence; and an oligonucleotide set, comprising a first oligonucleotide, a second oligonucleotide, a third oligonucleotide, and a fourth oligonucleotide, wherein: a 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide; (b) ligating: a 3′ terminus of the first oligonucleotide to a 5′ terminus of the first strand, a 5′ terminus of the second oligonucleotide to a 3′ terminus of the second strand, a 5′ terminus of the third oligonucleotide to a 3′ terminus of the first strand, and a 3′ terminus of the fourth oligonucleotide to a 5′ terminus of the second strand; and (c) performing extension reactions, comprising: extending the 3′ terminus of the second oligonucleotide using, in order, a portion of the third oligonucleotide, the first strand and the first oligonucleotide as a template, extending the 3′ terminus of the third oligonucleotide using, in order, a portion of the second oligonucleotide, the second strand, and the fourth oligonucleotide as a template, wherein the second oligonucleotide is crosslinked to the third oligonucleotide. In some implementations, the second oligonucleotide is crosslinked to the third oligonucleotide through a reversible crosslinker. In some implementations, the second oligonucleotide is crosslinked to the third oligonucleotide before the ligating. Alternatively, the second oligonucleotide is crosslinked to the third oligonucleotide after the ligating. In some implementations, the method further comprises reversing a crosslink between the second oligonucleotide and the third oligonucleotide.

Also described herein is a method for sequencing, comprising: (a) providing a nucleic acid molecule comprising, in order, a first sequence, a second sequence, and a third sequence, wherein the first sequence and the third sequence are identical; (b) sequencing the first sequence by, for each of a plurality of first flow steps in a plurality of first flow cycles, (i) providing labeled nucleotides of a single base type to a primer hybridized to the nucleic acid molecule, and (ii) detecting one or more signals indicative of incorporation, or lack thereof, of a labeled nucleotide in the primer; (c) processing the second sequence by, for at least one of a plurality of second flow steps in a plurality of second flow cycles, providing nucleotides of two or three base types to the primer; and (d) sequencing the third sequence by, for each of a plurality of third flow steps in a plurality of third flow cycles, (i) providing labeled nucleotides of a single base type to the primer, and (ii) detecting one or more signals indicative of incorporation, or lack thereof, of a labeled nucleotide in the primer. In some implementations, the labeled nucleotides provided in (b) or (d) are non-terminated. In some implementations, the nucleotides provided in (c) are non-terminated. In some implementations, the plurality of first flow cycles and the plurality of third flow cycles follows a first flow order, wherein the plurality of second flow cycles follows a second flow order different from the first flow order.

Further described herein is a method for sequencing, comprising: (a) providing a nucleic acid molecule comprising, in order, a first sequence, a second sequence, and a third sequence, wherein the first sequence is a copy of the third sequence except that (1) at least one base corresponding to a cytosine base in the third sequence is a thymine in the first sequence, or (2) at least one base corresponding to a guanine base in the third sequence is an adenine in the first sequence; (b) sequencing the first sequence by, for each of a plurality of first flow steps in a plurality of first flow cycles, (i) providing labeled nucleotides of a single base type to a primer hybridized to the nucleic acid molecule, and (ii) detecting one or more signals indicative of incorporation, or lack thereof, of a labeled nucleotide in the primer; (c) processing the second sequence by, for at least one of a plurality of second flow steps in a plurality of second flow cycles, providing nucleotides of two or three base types to the primer; and (d) sequencing the third sequence by, for each of a plurality of third flow steps in a plurality of third flow cycles, (i) providing labeled nucleotides of a single base type to the primer, and (ii) detecting one or more signals indicative of incorporation, or lack thereof, of a labeled nucleotide in the primer.

Also described is a method for sequencing, comprising: (a) providing a nucleic acid molecule comprising a first sequence and a second sequence, wherein the first sequence and the second sequence are identical; (b) sequencing the first sequence by, for each cycle of a plurality of first flow cycles, (i) providing labeled nucleotides of a first combination of three base types to a primer hybridized to the nucleic acid molecule, (ii) detecting one or more signals indicative of incorporation, or lack thereof, of one or more labeled nucleotides of the first combination of three base types in the primer, and (iii) providing nucleotides of a fourth base type different from the three base types in the first combination; and (c) sequencing the second sequence by, for each cycle of a plurality of second flow cycles, (i) providing labeled nucleotides of a second combination of three base types to the primer, wherein the second combination is different from the first combination, (ii) detecting one or more signals indicative of incorporation, or lack thereof, of one or more labeled nucleotides of the second combination of three base types in the primer, and (iii) providing nucleotides of a fifth base type different from the three base types in the second combination. In some implementations, the labeled nucleotides provided in (b) or (c) are non-terminated. In some implementations, the labeled nucleotides provided in (b) and (c) are non-terminated. In some implementations, nucleotides of the fourth base type provided in step (b) are labeled, and step (b) further comprises (iv) detecting one or more additional signals indicative of incorporation, or lack thereof, of one or more additional labeled nucleotides of the fourth base type. In some implementations, nucleotides of the fifth base type provided in step (c) are labeled, and step (c) further comprises (iv) detecting one or more additional signals indicative of incorporation, or lack thereof, of one or more additional labeled nucleotides of the fifth base type. In some implementations, the method further comprises comparing, or combining, first sequencing data corresponding to the one or more signals detected in step (b) and second sequencing data corresponding to the one or more signals detected in step (c), to determine at least a portion of the first sequence.

Further described herein is a method for processing a nucleic acid, comprising: performing extension reactions, in the presence of a mutagenesis agent, on a partially circular nucleic acid molecule comprising a first strand comprising a first template sequence and a second strand comprising a second template sequence, wherein the first template sequence is a reverse complement of the second template sequence, thereby generating a nucleic acid molecule comprising: a first strand comprising the first template sequence and a first copy portion, wherein the first copy portion is a copy of the first template sequence except that at least 1 base is different due to mutagenesis; and a second strand comprising the second template sequence and a second copy portion, wherein the second copy portion is a copy of the second template sequence except that at least 1 base is different due to mutagenesis.

In some implementations, the mutagenesis agent comprises one or more agents selected from the group consisting of: 8-oxo-dGTP, dPTP, 8-oxo-dG (8-oxo-2′-deoxyguanosine), 5Br-dUTP, 20H-dATP, and dITP. In some implementations, the mutagenesis agent induces one or more mutations selected from the group consisting of: A:T to C:G, T:A to G:C, A:T to T:A, A:T to G:C, G:C to A:T, T:A to C:G, and G:C to T:A. In some implementations, the first copy portion is a copy of the first template sequence except that at least 5 bases are different due to mutagenesis. In some implementations, the first copy portion is a copy of the first template sequence except that at least 10 bases are different due to mutagenesis.

In some implementations, the method further comprises amplifying the nucleic acid molecule. In some implementations, the method further comprises sequencing the nucleic acid molecule, or derivative thereof. In some implementations, the method further comprises determining data indicative of the length of a homopolymer sequence in the first template sequence based at least in part on processing two or more of first sequencing data corresponding to the first template sequence, second sequencing data corresponding to the first copy portion, third sequencing data corresponding to the second template sequence, and fourth sequencing data corresponding to the second copy portion.

Further described herein is a method, comprising: performing extension reactions, in the presence of deoxyuridine at a concentration of up to 10% of all nucleotides, on a partially circular nucleic acid molecule comprising a first strand comprising a first template sequence and a second strand comprising a second template sequence, wherein the first template sequence is a reverse complement of the second template sequence, thereby generating a nucleic acid molecule comprising: a first strand comprising the first template sequence and a first copy portion, wherein the first copy portion is a copy of the first template sequence except that at least base corresponding to a thymine in the first template sequence is a deoxyuridine; and a second strand comprising the second template sequence and a second copy portion, wherein the second copy portion is a copy of the second template sequence except that at least base corresponding to a thymine in the second template sequence is a deoxyuridine. In some implementations, the method further comprises subjecting the nucleic acid molecule to a cleavage reaction at one or more deoxyuridine sites, to generate a truncated molecule. In some implementations, the method further comprises digesting single strand deoxyribonucleic acid (DNA) of the truncated molecule, to generate a second truncated molecule. In some implementations, the digesting is performed by an exonuclease. In some implementations, the method further comprises coupling one or more adapters to the second truncated molecule.

Also described herein is a targeted capture method, comprising: providing a nucleic acid molecule comprising, in the same strand, a template sequence and a copy sequence, wherein the copy sequence is a copy of the template sequence except that substantially all cytosine bases in the copy sequence are methylated; converting unmethylated cytosine residues in the nucleic acid molecule to uracil residues, thereby generating a converted nucleic acid molecule comprising the copy sequence and a converted template sequence; hybridizing a capture probe to at least a portion of the copy sequence. The method may further include amplifying the converted nucleic acid molecule, thereby substituting uracil residues in the converted template sequence with thymine residues to form an amplicon, wherein the capture probe hybridizes to at least a portion of the copy sequence in the amplicon. The template sequence may be in a 5′ portion of the nucleic acid molecule relative to the copy sequence. Further, the converted template sequence may be in a 5′ portion of the converted nucleic acid molecule relative to the copy sequence.

The targeted capture method may further include sequencing the converted template sequence without sequencing the copy sequence.

The capture probe used in the targeted capture may include a capture sequence configured to target a CpG site in the copy sequence. In some implementations, the capture sequence is at least 20 bases in length. In some implementations, the capture sequence is at least 50 bases in length. In some implementations, the capture sequence is at least 80 bases in length.

The targeted capture method may be applied to a pool of nucleic acid molecules. For example, the method can include providing a plurality of nucleic acid molecules, each comprising, in the same strand, a template sequence and a copy sequence, wherein the copy sequence is a copy of the template sequence except that substantially all cytosine bases in the copy sequence are methylated, wherein a first portion of nucleic acid molecules in the plurality of nucleic acid molecules comprises a different template sequence than a second portion of nucleic acid molecules in the plurality of nucleic acid molecules; converting unmethylated cytosine residues in the plurality of nucleic acid molecules to uracil residues, thereby generating a plurality of converted nucleic acid molecules, each converted nucleic acid molecule comprising the copy sequence and a converted template sequence; and hybridizing a plurality of capture probes to at least a portion of the copy sequences. The method may further include amplifying the plurality of converted nucleic acid molecules, thereby substituting uracil residues in the converted template sequence with thymine residues to form a plurality of amplicons, wherein the capture probes hybridize to at least a portion of the copy sequence in at least a portion of the amplicons. The method may further include separating amplicons hybridized to capture probes from amplicons that are not hybridized to capture probes.

The targeted capture method may further include generating the nucleic acid molecule using a nucleic acid sample obtained from a subject. For example, the nucleic acid molecule may be generated by performing extension reactions, in the presence of a nucleotide reagent comprising methylated cytosine bases methylated cytosine, on a partially circular nucleic acid molecule comprising a first strand comprising the template sequence and a second strand comprising a second template sequence, wherein the template sequence is a reverse complement of the second template sequence, thereby generating a nucleic acid molecule comprising: a first strand comprising the template sequence and the copy sequence; and a second strand comprising the second template sequence and a second copy sequence, wherein the second copy portion is a copy of the second template sequence except that substantially all cytosine bases in the second copy portion are methylated. In some implementations, substantially all cytosine bases in the nucleotide reagent are methylated cytosine bases. In some implementations, the template sequence or the second template sequence comprises at least one non-methylated cytosine. For example, the nucleic acid molecule may be made by providing: a template nucleic acid molecule comprising a first strand comprising template sequence and a second strand comprising the second template sequence; and an oligonucleotide set, comprising a first oligonucleotide, a second oligonucleotide, a third oligonucleotide, and a fourth oligonucleotide, wherein: a 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide; ligating: a 3′ terminus of the first oligonucleotide to a 5′ terminus of the first strand, a 5′ terminus of the second oligonucleotide to a 3′ terminus of the second strand, a 5′ terminus of the third oligonucleotide to a 3′ terminus of the first strand, and a 3′ terminus of the fourth oligonucleotide to a 5′ terminus of the second strand; and performing extension reactions in the presence of a nucleotide reagent comprising methylated cytosine bases, comprising: extending the 3′ terminus of the second oligonucleotide using, in order, a portion of the third oligonucleotide, the first strand, and the first oligonucleotide as a template, extending the 3′ terminus of the third oligonucleotide using, in order, a portion of the second oligonucleotide, the second strand, and the fourth oligonucleotide as a template.

### BRIEF DESCRIPTION OF THE DRAWINGS

FIG. 1 illustrates an exemplary embodiment of a nucleic acid construct described herein.

FIG. 2 shows an exemplary method of making a nucleic acid construct used according to the methods described herein.

FIG. 3 illustrates exemplary methylation status data that may be obtained using the method described herein.

FIG. 4 illustrates an exemplary method of making a construct for pseudo paired end sequencing.

FIG. 5A illustrates an exemplary method for obtaining methylation profiling data for a nucleic acid molecule.

FIG. 5B shows an exemplary method for generating methylation profiling data in accordance with some embodiments.

FIG. 6 shows an exemplary method for generating methylation profiling data in accordance with some embodiments.

FIG. 7 shows an exemplary method for targeted enrichment of a CpG site according to some embodiments.

### DETAILED DESCRIPTION OF THE INVENTION

Described herein are compositions, including nucleic acid constructs, that may be used for methylation sequencing. Also described are methods of making such nucleic acid constructs and compositions, as well as analyzing, for example by sequencing, the same.

### Definitions

As used in this specification and the appended claims, the singular forms “a”, “an” and “the” include plural references unless the context clearly dictates otherwise.

Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “about” a number refers to that number plus or minus 10% of that number. The term “about” when used in the context of a range refers to that range minus 10% of its lowest value and plus 10% of its greatest value.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

As used herein, the term “nucleotide” refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a nucleotide analog that is a modified, synthesized, or engineered nucleotide). A naturally occurring nucleotide may include a canonical base (e.g., A, C, G, T, or U). A nucleotide analog may not be naturally occurring or may include a non-canonical base (e.g., an alternative base). The nucleotide analog may include a modified polyphosphate chain (e.g., triphosphate coupled to a fluorophore). The nucleotide analog may comprise a label. The nucleotide analog may be terminated (e.g., reversibly terminated). Nucleotide analogs that may be used in accordance with embodiments of this disclosure are described, for example, in United States Patent Publication No. 2021/0230669, which is hereby incorporated by reference in its entirety.

The terms “label,” “tag,” or “dye” are used interchangeably herein, and generally refer to a moiety that is capable of coupling with a species, such as, for example a nucleotide analog. A label may include an affinity moiety. In some cases, a label may be a detectable label that emits a signal (or reduces an already emitted signal) that can be detected (e.g., a fluorescent tag). In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), or tris(hydroxypropyl)phosphine (THP)), or enzymatically cleavable (e.g., via an esterase, lipase, peptidase, or protease). As disclosed herein, the terms cleavable and excisable are used interchangeably. In some cases, the label may be luminescent, that is, fluorescent or phosphorescent. Labels may be quencher molecules. Dyes, quenchers, and labels may be incorporated into nucleic acid sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410).

As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity as such sequences would generally be considered “identical”.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(e.g., [A T G C A T G C A T G A T G A T G A T G C A T G C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order and can be expressed in units of flow cycles. For example, where [A T G C] is identified as a 1st flow cycle, and [A T G] is identified as a 2nd flow cycle, the flow order of [A T G C A T G C A T G A T G A T G A T G C A T G C] may be described as having a flow-cycle order of [1st flow cycle; 1st flow cycle; 2nd flow cycle; 2nd flow cycle; 2nd flow cycle; 1st flow cycle; 1st flow cycle]. Alternatively or in addition, the flow cycle order may be described as [cycle 1, cycle, 2, cycle 3, cycle 4, cycle 5, cycle 6], where cycle 1 is the 1st flow cycle, cycle 2 is the 1st flow cycle, cycle 3 is the 2nd flow cycle, etc.

It is understood that aspects and variations of the invention described herein include “consisting of” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined; the order of some blocks is, optionally, changed; and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Nucleic Acid Constructs

A nucleic acid construct that may be used in accordance with the methods described herein can include a first nucleic acid strand and a second nucleic acid strand, which may hybridize to each other (e.g., in water at 25° C.). The first and second nucleic acid strands can be derived from a nucleic acid duplex, which may be obtained from patient sample(s). For example, the nucleic acid duplex may be a DNA fragment from a tissue sample or a cell-free DNA (cfDNA) sample. The first strand of the construct can correspond to the “top” strand of the nucleic acid duplex, and the second strand of the construct can correspond to the “bottom” strand of the nucleic acid duplex. The nucleic acid duplex can include a first template sequence in the top strand and a second template sequence in the bottom strand, and the template sequences are used to generate the nucleic acid construct. The first strand of the nucleic acid construct can include two copies of the first template sequence, which may be identical copies or may differ based on the methylation profile of the first template sequence (for example, if used in a method to determine the methylation profile of the first template sequence as described herein). Similarly, the second strand of the nucleic acid construct can include two copies of the second template sequence, which may be identical copies or may differ based on the methylation profile of the second template sequence (for example, if used in a method to determine the methylation profile of the second template sequence).

The nucleic acid construct may be synthesized in the presence of nucleotides (e.g., deoxynucleotides) that include 5-methylcytosine (5mC) in place of canonical cytosine (e.g., A, T, G, and 5mC, and excluding C), such that the resulting nucleic acid construct includes a first portion (i.e., corresponding to the first template sequence with the original methylation profile) and a first copy portion, wherein the first copy portion is a copy of the first portion except that substantially all cytosine bases in the first copy portion are methylated (i.e., 5-methylcytosine); and a second strand comprising a second portion (i.e., corresponding to the second template sequence with the original methylation profile) and a second copy portion, wherein the second copy portion is a copy of the second portion except that substantially all cytosine bases in the second copy portion are methylated (i.e., 5-methylcytosine). The first and second portions may therefore include methylated cytosine (i.e., naturally occurring methylated cytosine) and non-methylated cytosine (i.e., naturally occurring non-methylated cytosine), while the first and second copy portions include all methylated cytosine (i.e., 5-methylcytosine). The first portion has sequence homology to the first copy portion (except for methylation profile), and the second portion has sequence homology to the second copy portion (except for methylation profile).

The nucleic acid construct be subjected to a conversion reaction, wherein non-methylated cytosine is converted to uracil. If the first (or second) copy portion includes only methylated cytosine, the sequence of the first (or second) copy portion is not modified and remains identical to the original first (or second) template strand. If the first (or second) portion, however, includes both methylated and non-methylated cytosine, then the conversion reaction will alter the sequence of the first (or second) portion such that substantially all of the non-methylated cytosine bases in the first (or second) portion become uracil bases. “Substantially all” in this context indicates that the conversion reaction may be incomplete such that a small portion (e.g., less than 10%) of non-methylated cytosine may remain as non-methylated cytosine bases. In some cases, subsequent to the conversion reaction, at most about 10.0%, 9.5%, 9.0%, 8.5%, 8.0%, 7.5%, 7.0%, 6.5%, 6.0%, 5.5%, 5.0%, 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, 0.10%, or less of non-methylated cytosine bases in the first (or second) portion remain as non-methylated cytosine bases. The resulting converted nucleic acid construct thus comprises i) a first strand comprising a first portion and a first copy portion, wherein the first copy portion is a copy of the first portion except that substantially all cytosine bases in the first copy portion are methylated cytosine, and substantially all bases in the first portion that correspond to cytosine bases in the first copy portion are methylated cytosine or uracil and ii) a second strand comprising a second portion and a second copy portion, wherein the second copy portion is a copy of the second portion except that substantially all cytosine bases in the second copy portion are methylated cytosine, and substantially all bases in the second portion that correspond to cytosine bases in the second copy portion are methylated cytosine or uracil.

The resulting nucleic acid construct may be amplified (e.g., through PCR amplification, multiple displacement amplification, etc.) in the presence of canonical deoxynucleotides (A, G, C, T), which amplification replaces any uracil bases with thymine bases. Thus, the amplified nucleic acid construct comprises a first strand comprising a first portion and a first copy portion. The first copy portion is a copy of the first portion, except that i) substantially all cytosine bases in the first copy portion are methylated cytosine, and ii) substantially all bases in the first portion that correspond to cytosine bases in the first copy portion are methylated cytosine or thymine. The amplified nucleic acid construct further comprises a second strand comprising a second portion and a second copy portion, wherein the second copy portion is a copy of the second portion except that substantially all cytosine bases in the second copy portion are methylated cytosine, and substantially all bases in the second portion that correspond to cytosine bases in the second copy portion are methylated cytosine or thymine. That is, nearly all cytosine bases in the first copy portion and the second copy portion are methylated cytosines.

Alternatively, the nucleic acid construct may be synthesized in the presence of only canonical nucleotides (e.g., deoxynucleotides) (e.g., A, T, C, and G, with no methylated cytosine nucleotides available for synthesis). In such cases, the resulting nucleic acid construct includes a first strand comprising a first portion and a first copy portion, wherein the first copy portion is a copy of the first portion except that all cytosine bases in the first copy portion are non-methylated; and a second strand comprising a second portion and a second copy portion, wherein the second copy portion is a copy of the second portion except that all cytosine bases in the second copy portion are non-methylated. The construct may be subjected to a conversion reaction wherein methylated cytosine is converted to uracil, which provides a converted nucleic acid construct that includes a first strand comprising a first portion and a first copy portion, wherein the first copy portion is a copy of the first portion except that at least a portion of bases in the first portion that correspond to cytosine bases in the first copy portion are uracil; and a second strand comprising a second portion and a second copy portion, wherein the second copy portion is a copy of the second portion except that at least a portion of bases in the second portion that correspond to cytosine bases in the second copy portion are uracil. Cytosine bases in the first (or second) copy portion remain cytosine when the construct is synthesized using non-methylated cytosine. The nucleic acid construct may be amplified (e.g., through PCR amplification) in the presence of canonical deoxynucleotides (A, G, C, T), which replaces the uracil bases with thymine bases. Thus, the amplified nucleic acid construct includes a first strand comprising a first portion and a first copy portion, wherein the first copy portion is a copy of the first portion except that at least a portion of bases in the first portion that correspond to cytosine bases in the first copy portion are thymine; and a second strand comprising a second portion and a second copy portion, wherein the second copy portion is a copy of the second portion except that at least a portion of bases in the second portion that correspond to cytosine bases in the second copy portion are thymine. Cytosine bases in the first (or second) portion that were not methylated in the original first (or second) template (e.g., the first or second portion) are not converted, and thus remain as cytosine bases. Accordingly, at least one cytosine base in the first portion or the second portion is not methylated.

When the first or second copy portions are synthesized using methylated cytosine (i.e., omitting non-methylated cytosine) and non-methylated cytosine is converted to uracil or thymine, or alternatively when the first or second copy portions are synthesized using non-methylated cytosine (i.e., omitting methylated cytosine) and methylated cytosine is converted to uracil or thymine, the first and second copy portion retain the sequence of the first and second template sequences, respectively. Thus, when the first and second template sequences are reverse complements of each other (for example, when they are a nucleic acid duplex from a biological sample of a subject), the first copy portion is a reverse complement of the second copy portion.

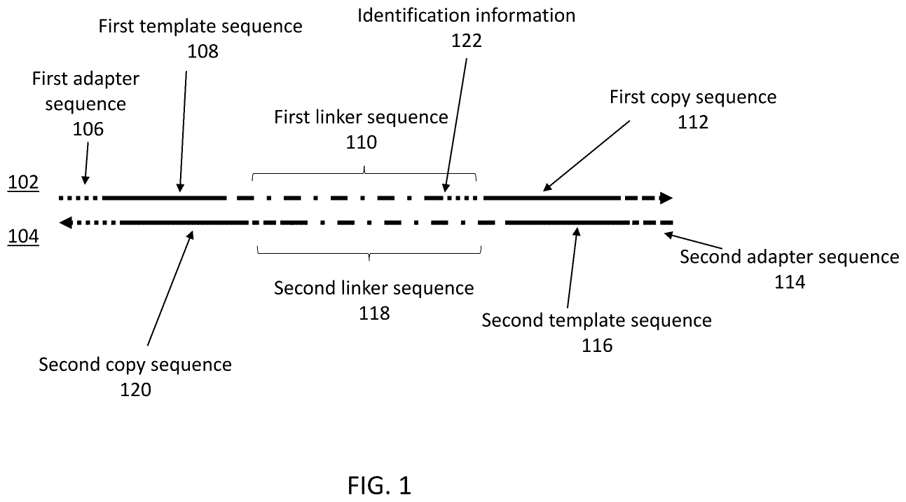

The first portion and the first copy portion of the first strand in the nucleic acid construct may be separated by a first nucleic acid linker. Similarly, the second portion and the second copy portion of the second strand in the nucleic acid construct may be separated by a second nucleic acid linker. See e.g., FIG. 1, where a region between the first template sequence 108 and the first copy sequence 112 comprises the first linker sequence 110. The first nucleic acid linker and the second nucleic acid linker may be reverse complements of each other. For example, the first nucleic acid linker and the second nucleic acid linker may be synthesized using the construct synthesis methods described herein. Optionally, the linker can include identification information, such as a unique molecular identifier (UMI) and/or a sample barcode (also known as a “sample index”). The identification information can help trace the original duplex nucleic acid molecule obtained from the biological sample (i.e., for the UMI) or the sample of origin when multiple samples are pooled together and simultaneously sequenced (i.e., for the sample barcode).

A linker is not a region of interest for sequencing, and as such the linker sequence or length may be chosen to reduce the amount of effort required to sequence through the linker. In some cases, the linker sequence may be predetermined. For example, a linker sequence may be selected based on flow-cycle order (e.g., the order of nucleic acid bases used for sequencing). Alternatively, a linker sequence or portions thereof may be random. In some cases, alternatively or additional, a linker sequence or portions thereof may be selected based on predicted structural features of the sequence. Particular sequences or sequence repeats are known in the art to produce structural changes to a nucleic acid molecule. For instance, A:T tracts (e.g., at least four A:T base pairs in a row) have an intrinsically bent structure and also induce bending within an encompassing oligonucleotide. See e.g., Martin-Gonzalez et al. Understanding the paradoxical mechanical response of in-phase A-tracts at different force regimes. Nucl Acids Res 48(9), 5024-5036 (2020); and Largy and Mergny Shape matters: size-exclusion HPLC for the study of nucleic acid structural polymorphism. Nucl Acids Res 42(19), e149 (2014). Other structure-influencing sequences as known in the art may also be used to produce desired feature in a linker.

The linker may be derived during synthesis of the nucleic acid construct, which can rely on an extension reaction performed on partially circularized nucleic acid as further described herein. The linker may be long enough to allow for an appropriate curvature of the partially circularized nucleic acid while still allowing a template sequence to function as a template during the extension reaction. In some implementations, the first nucleic acid linker and/or second nucleic acid linker is about 30 bases in length or more (e.g., about 40 bases in length or more, about 50 bases in length or more, about 60 bases in length or more, about 70 bases in length or more, about 80 bases in length or more, about 90 bases in length or more, or about 100 bases in length or more). The linker length may be set to a maximum length to avoid over-winding of the nucleic acid molecule. The maximum length may depend on the length of the template. For example, in some implementations, the first nucleic acid linker and/or second nucleic acid linker is about the length the first portion or the second portion or less. In some implementations, the first nucleic acid linker and/or second nucleic acid linker is between about 20% and about 100% (e.g., about 20% to about 30%, about 30% to about 40%, about 40% to about 50%, about 50% to about 60%, about 60% to about 70%, about 70% to about 80%, about 80% to about 90%, or about 90% to about 100%) of a length of the first portion or the second portion.

The nucleic acid construct may include sequencing adapter sequences that include a hybridization site for a sequencing primer. For example, the first strand can include a first sequencing adapter sequence, and the second strand can include a second sequencing adapter sequence. The sequencing adapter may be proximal to the 3′ end (i.e., relative to the portion(s) and copy portion(s), and linker if present) of the first or second strand of the nucleic acid construct. The sequencing adapter sequences may be the same nucleic acid sequence. Optionally, the sequencing adapter sequence(s) can include identification information, such as a unique molecular identifier (UMI) and/or a sample barcode (also known as a “sample index”).

FIG. 1 illustrates an exemplary embodiment of a nucleic acid construct described herein. The construct includes a top strand (i.e., first strand) 102 and a bottom strand (i.e., second strand) 104. The first strand 102 includes, from 5′ to 3′, a first sequencing adapter sequence 106, a first template sequence 108, a first linker sequence 110, and a first copy sequence 112. The second strand 104 includes, from 5′ to 3′, a second sequencing adapter sequence 114, a second template sequence 116, a second linker sequence 118, and a second copy sequence 120. The first and second linker sequences 110 and 118 may be reverse complements of each other. The linker sequences 110 and 118 may optionally include identification information 122. Alternatively, the identification information 122 may be located in the first adapter sequence 106 or the second adapter sequence 114.

The nucleic acid construct may be synthesized using a concatenating synthesis process. For example, the construct may be synthesized by, or modified from a construct synthesized by, the method described in Bae et al., CODEC enables ‘single duplex’ sequencing, bioRxiv, no. 448110 (2021), the contents of which are incorporated by reference for all purposes. In some embodiments, the concatenating synthesis may be a rolling circle amplification (RCA) synthesis. Either method may be modified, in some embodiments, by performing the extension reaction in the presence of methylated cytosine (e.g., 5-mehtylcytotsine). Although the description below is provided in the context of synthesizing the nucleic acid construct in the presence of methylated cytosine, it is understood that, in some embodiments of the methods described herein, no methylated cytosine is present in the synthesis reaction.

For example, a method of making the nucleic acid construct can include performing extension reactions, in the presence of methylated cytosine (e.g., wherein substantially all or all cytosine bases present in the extension are methylated cytosine (e.g., 5-methycytosistine), on a partially circular nucleic acid molecule comprising a first strand comprising a first template sequence and a second strand comprising a second template sequence, wherein the first template sequence is a reverse complement of the second template sequence. The method thereby generates a nucleic acid molecule that includes a first strand comprising the first template sequence and a first copy portion, wherein the first copy portion is a copy of the first template sequence except that substantially all (or all) cytosine bases in the first copy portion are methylated; and a second strand comprising the second template sequence and a second copy portion, wherein the second copy portion is a copy of the second template sequence except that substantially all (or all) cytosine bases in the second copy portion are methylated.

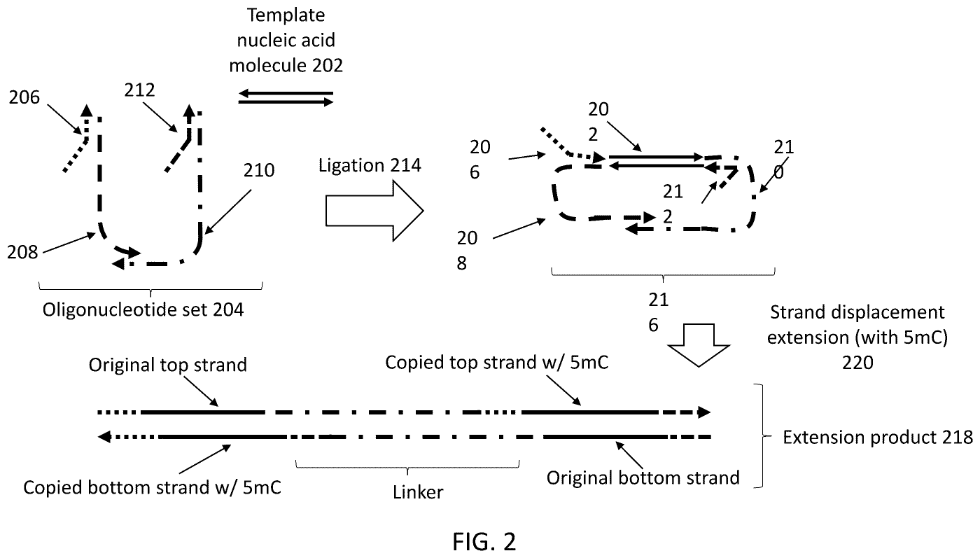

FIG. 2 shows an exemplary method of making a nucleic acid construct used according to the methods described herein. The nucleic acid construct may be synthesized by providing a template nucleic acid molecule 202 and an oligonucleotide set 204 of four oligonucleotides. The template nucleic acid may be, for example, the duplex nucleic acid molecule obtained from the biological sample form a subject. The template nucleic acid molecule includes a first strand comprising a first template sequence (i.e., corresponding to the first portion in the construct discussed above) and a second strand comprising a second template sequence (corresponding to the second portion in the construct discussed above). The template nucleic acid may have a naturally occurring methylation profile. The template nucleic acid may be prepared for construct synthesis, for example by nucleic acid end repair and/or A-tailing. The first strand and/or second strand of the nucleic acid molecule may be a cfDNA molecule.

The template nucleic acid molecule may be, in some embodiments, up to 100 bases (bp), 150 bp, 200 bp, 250 bp, 300 bp, 400 bp, 500 bp, 600 bp, 700 bp, 800 bp, 900 bp or 1,000 bp in length. In some embodiments, the length can be longer than 1,000 bp such as up to 1.1 kilobases (kb), 1.2 kb, 1.3 kb, 1.4 kb, 1.5 kb, 1.6 kb, 1.7 kb, 1.8 kb, 1.9 kb, or 2 kb or longer.

The template nucleic acid molecules used in the methods described herein may be obtained from any suitable biological source, for example a tissue sample, a blood sample, a serum sample, a cerebrospinal fluid sample, a plasma sample, a saliva sample, a fecal sample, or a urine sample. In some embodiments, RNA polynucleotides are reverse transcribed into DNA polynucleotides. In some embodiments, the polynucleotide is a cell-free DNA (cfDNA), such as a circulating tumor DNA (ctDNA) or a fetal cell-free DNA.

The oligonucleotide set 204 includes four oligonucleotides, portions of which hybridize (e.g., through reverse complementarity) to form a complex comprising the four-oligonucleotides. The following discussions refer to a “3′ portion” and a “5′ portion” of the oligonucleotide. The 3′ and 5′ portion is to indicate the proximal location of the referenced portion, although the referenced portion need not be at the 3′ terminus or 5′ terminus, respectively, of the oligonucleotide. In some implementations, the referenced 3′ or 5′ portion is within 10, 9, 8, 7, 6, 5, 4, 3, 2, or 1 bases of the 3′ or 5′ terminus, or may be at the 3′ terminus or 5′ terminus. The oligonucleotide set can assemble such that a 3′ portion of the first oligonucleotide 206 hybridizes to a 5′ portion of the second oligonucleotide 208, a 3′ portion of the second oligonucleotide 208 hybridizes to a 3′ portion of the third oligonucleotide 210, and a 5′ portion of the third oligonucleotide 210 hybridizes to a 3′ portion of the fourth oligonucleotide 212. The first oligonucleotide may further include a 5′ portion that include adapter sequence (e.g., includes a hybridization site for a sequencing primer). Similarly, the second oligonucleotide may include a 5′ portion that include adapter sequence (e.g., includes a hybridization site for a sequencing primer), which may be the same or different as the adapter sequence included in the first oligonucleotide.

Optionally, the second oligonucleotide 208 is cross-linked to the third oligonucleotide 210 through a crosslinker, which may be a reversible crosslinker. Exemplary reversible crosslinkers include a psoralen crosslinker or a 3-cyanovinylcarbazole (CNVK) crosslinker. Other reversible crosslinkers are known in the art. The crosslinker can crosslink the portion of the second oligonucleotide that hybridizes to the portion of the third oligonucleotide. For example, the 3′ portion of the second oligonucleotide can include a first member of a crosslinker (e.g., a reversible crosslinker) and the 3′ portion of the third oligonucleotide can include a second member of the crosslinker.

Optionally, the first oligonucleotide 206 is cross-linked to the second oligonucleotide 208 through a crosslinker, which may be a reversible crosslinker. The crosslinker can crosslink the portion of the first oligonucleotide that hybridizes to the portion of the second oligonucleotide. For example, the 3′ portion of the first oligonucleotide can include a first member of a crosslinker (e.g., a reversible crosslinker) and the 5′ portion of the second oligonucleotide can include a second member of the crosslinker.

Optionally, the third oligonucleotide 210 is cross-linked to the fourth oligonucleotide 212 through a crosslinker, which may be a reversible crosslinker. The crosslinker can crosslink the portion of the third oligonucleotide that hybridizes to the portion of the fourth oligonucleotide. For example, the 5′ portion of the third oligonucleotide can include a first member of a crosslinker (e.g., a reversible crosslinker) and the 3′ portion of the fourth oligonucleotide can include a second member of the crosslinker.

The crosslinker between the first and second oligonucleotides may be of a same type as the crosslinker between the second and third oligonucleotides. The crosslinker between the third and fourth oligonucleotides may be of a same type as the crosslinker between the second and third oligonucleotides and/or the crosslinker between the first and the second oligonucleotides. It is advantageous in cases where crosslinkers are used between more than one pair of oligonucleotides (e.g., between the first and second oligonucleotides and between the second and third oligonucleotides) for the crosslinkers to be of a same type. Then only a single reaction step may be required for reversing the crosslinking between the pairs of oligonucleotides).

Crosslinking between one or more pairs of oligonucleotides may improve overall ligation efficiency between the oligonucleotide set and the template nucleic acid molecule. In some cases, crosslinking between one or more pairs of oligonucleotides may improve overall ligation efficiency by at least 1%, at least 2%, at least 3%, at least 4%, at least 5%, at least 6%, at least 7%, at least 8%, at least 9%, at least 10%, at least 15%, at least 20%, at least 25%, or at least 30% (e.g., as compared to ligation efficiency between a non-crosslinked oligonucleotide set and the template nucleic acid molecule).

The oligonucleotide set is then ligated to the template nucleic acid at 214. For example, a 3′ terminus of the first oligonucleotide can be ligated to a 5′ terminus of the first strand of the template nucleic acid, a 5′ terminus of the second oligonucleotide can be ligated to a 3′ terminus of the second strand, a 5′ terminus of the third oligonucleotide can be ligated to a 3′ terminus of the first strand, and a 3′ terminus of the fourth oligonucleotide can be ligated to a 5′ terminus of the second strand. In some implementations, the second oligonucleotide is cross-linked to the third oligonucleotide prior to the ligating. In some implementations, the second oligonucleotide is cross-linked to the third oligonucleotide after the ligating. The resulting nucleic acid construct is a partially circular nucleic acid molecule 216 that includes a first strand comprising a first template sequence and a second strand comprising a second template sequence, wherein the first template sequence is a reverse complement of the second template sequence.

An extension reaction 220 is then performed on the partially circular nucleic acid molecule. The 3′ terminus of the second oligonucleotide is extended using, in order, a portion of the third oligonucleotide, the first strand and the first oligonucleotide as a template. The 3′ terminus of the third oligonucleotide is also extended using, in order, a portion of the second oligonucleotide, the second strand, and the fourth oligonucleotide as a template. In some implementations, the extension reactions occur in the presence of a nucleotide reagent that includes methylated cytosine bases (e.g., 5-methylcytosine). For example, substantially all cytosine bases in the nucleotide reagent may be methylated cytosine bases. The nucleotide regent also includes other nucleotides necessary for the extension reaction (e.g., A, T, and G bases). In some implementations, the optional reversible crosslinker is reversed after the extension reactions. The resulting nucleic acid construct 218 includes a first strand comprising the first template sequence portion (“original top”) and a first copy portion (“copied top”), and a second strand comprising the second template sequence portion (“original bottom”) and a second copy portion (“copied bottom”).

Once the nucleic acid construct is generated through the extension reaction, non-methylated cytosine in the construct may be converted to uracil. Conversion may be chemical or enzymatic. For example, in some embodiments, the nucleic acid constructs are treated with bisulfite to convert non-methylated cytosine to uracil. Alternatively, an enzymatic method may be used, for example by treating the nucleic acid construct with an enzyme that converts non-methylated cytosine to uracil, for example using NEBNext® Enzymatic Methyl-seq Kit (New England BioLabs), a ten-eleven translocation methylcytosine dioxygenase 2 (TET2) enzyme, or an APOBEC2 enzyme. Alternatively, methylated cytosine in the construct may be converted to uracil. See, for example, Liu et al., Bisulfate-free direct detection of 5-methylcytosine and 5-hydroxymethylcytosine at base resolution, Nature Biotechnology, vol. 37, pp. 424-429 (2019). This process of converting non-methylated cytosine to uracil (or, alternatively, methylated cytosine to uracil) results in a converted nucleic acid molecule comprising a first converted strand comprising a first converted template portion and a first converted copy portion, or a second converted strand comprising a second converted template portion and a second converted copy portion.

The converted nucleic acid molecule may be amplified. Amplification may occur in the presence of canonical deoxynucleotides (e.g., A, C, T, and G, excluding methylated cytosine), which cause uracil in the converted nucleic acid construct to be replaced with thymine in the resulting amplicons. The resulting nucleic acid construct includes a first portion (corresponding to the first template sequence) and a first copy portion, wherein the first portion and the first copy portion differ based on the methylation profile of the first template sequence. The construct also includes a second portion (corresponding to the second template sequence) and a second copy portion, wherein the second portion and the second copy portion differ based on the methylation profile of the second template sequence.

The nucleic acid constructs described herein may be sequenced, for example to determine a methylation profile of the first template sequence and/or a methylation profile of the second template sequence. That is, the difference between the sequence of the first portion and the first copy portion can indicate the methylation profile of the first template sequence, and the difference between the sequence of the second portion and the second copy portion can indicate the methylation profile of the second template sequence.

### Targeted Capture

Capture probes may be used to enrich for targeted sequences (e.g., targeted CpG sequences) prior to sequencing. Pools of sequencing constructs formed from template nucleic acid molecules, e.g., those obtained from a sample from a subject, may include many template sequence of low interest (for example, templates sequences that include no CpG methylation sites, or are otherwise from a region of the genome that is of low interest). Thus, sequencing all template sequences in the pool can result in unnecessary sequencing throughput, which uses additional reagents and analytical power for interpreting the sequencing data.

To enrich for targeted sequences, a pool of converted constructs (e.g., after completing a non-methylated cytosine to uracil conversion reaction, or after an amplification reaction to convert uracil to thymine residues) can be contacted with a plurality of capture probes. The capture probes can include a capture sequence (i.e., a nucleotide sequence) configured to target a region (e.g., CpG site) in the original template sequence (i.e., prior to conversion). The targeted region may be a predetermined CpG site, for example a CpG site from within a selected gene. The capture sequence may be, for example, at least 10 bases in length, at least 20 bases in length, at least 30 bases in length, at least 40 bases in length, at least 50 bases in length, at least 60 bases in length, at least 70 bases in length, at least 80 bases in length, at least 90 bases in length, at least 100 bases in length or longer. In addition to the capture sequence, the capture probe may optionally include a 5′ and/or 3′ flanking region, which does not hybridize to the targeted sequence. The capture probe may also include a binding moiety (e.g., biotin), which can be used to separate nucleic acid molecules hybridized to the capture probe from those that do not hybridize (or have not hybridized) to the capture probe.

The capture probes may be mixed with the pool of nucleic acid molecule constructs after amplification of the nucleic acid molecule constructs. This can help ensure that sufficient nucleic acid material is available for efficient capture. In some instances, e.g., where a biological sample obtained from a subject comprises a sufficiently large amount of nucleic acids, the capture probes may be mixed with the pool of nucleic acid molecule constructs prior to amplification of the constructs. This can help reduce any possible amplification bias in downstream sequencing results.

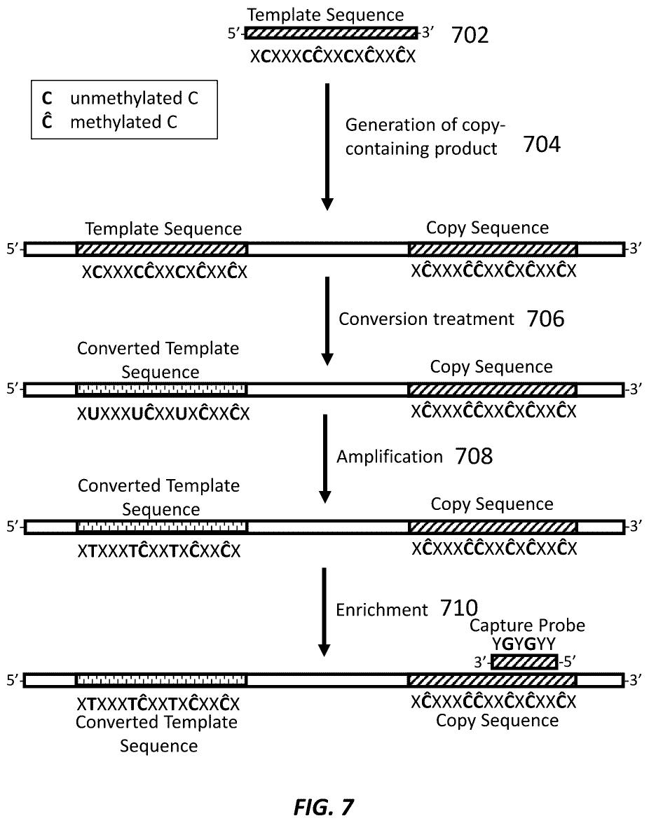

FIG. 7 shows an exemplary method for targeted enrichment of a CpG site according to some embodiments. At 702, a template nucleic acid molecule is provided, which includes a template sequence. The template sequence may include one or more CpG sites and/or include one or more methylated cytosine residues. The template sequence may include one or more unmethylated cytosine residues. The template nucleic acid molecule may be a duplex nucleic acid molecule. In some cases, the template nucleic acid molecule can include a second template sequence that is a reverse complement of the first template sequence. At 704, a nucleic acid molecule construct is generated, which includes the template sequence and a copy of the template sequence (i.e., a “copy sequence”), which sequences differ only in the methylation status of the cytosine residues. For example, the nucleic acid molecule construct may be generated in the presence of a nucleotide reagent that includes methylated cytosine bases (e.g., all or substantially all cytosine bases in the nucleotide reagent are methylated) such that when the nucleic acid molecule construct is generated, the cytosine residues in the copy sequence are all methylated or substantially all cytosine residues in the copy sequence are methylated.

The nucleic acid molecule construct formed at 704 may be made according to the methods described herein. For example, the template nucleic acid molecule may be combined with an oligonucleotide set comprising a first oligonucleotide, a second oligonucleotide, a third oligonucleotide, and a fourth oligonucleotide. A 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide. The oligonucleotide set may then be ligated to the template nucleic acid molecule. For example, a 3′ terminus of the first oligonucleotide can be ligated to a 5′ terminus of the first strand, a 5′ terminus of the second oligonucleotide can be ligated to a 3′ terminus of the second strand, a 5′ terminus of the third oligonucleotide can be ligated to a 3′ terminus of the first strand, and a 3′ terminus of the fourth oligonucleotide can be ligated to a 5′ terminus of the second strand. The ligation reaction thereby forms a partially circular nucleic acid molecule. After ligation, extension reactions can be performed in the presence of the nucleotide reagent that includes methylated cytosine bases to form the nucleic acid molecule construct.

At 706, unmethylated cytosine residues in the nucleic acid molecule are converted to uracil residues. This generates a converted nucleic acid molecule that includes the copy sequence (which is the same as the original template sequence, as cytosine bases in the copy sequence were methylated and therefore protected from the conversion reaction) and a converted template sequence, which includes cytosine bases (corresponding to methylated cytosine bases in the original template strand) and uracil bases (corresponding to unmethylated cytosine bases in the original template strand). The conversion reaction may be performed, for example, according to the methods described herein.

The converted nucleic acid construct may be amplified (e.g., through PCR amplification) in the presence of canonical deoxynucleotides (A, G, C, T) at 708. Amplification replaces any uracil bases with thymine bases in the resulting amplicon. Thus, the amplicons include a converted template sequence that includes cytosine nucleotides (corresponding to methylated cytosine nucleotides in the original template sequence) and thymine nucleotides (corresponding to unmethylated cytosine nucleotides and original thymine nucleotides in the original template sequence).

At 710, targeted template sequences are enriched. A capture probe configured to hybridize to at least a portion of the copy sequence is contacted with the amplicon, thus allowing the capture probe to hybridize to the amplicon. In some implementations, the capture probe may be contacted with the converted nucleic acid molecule, for example prior to amplification or in a method that does not include an amplification step. Because the converted template sequence differs from the copy sequence based on methylation status and conversion, the capture probe binds the copy sequence.

The capture probe may be designed such that it is agnostic to the original methylation status as a copy of the original sequence (prior to conversion) is conserved post-conversion. That is, the capture probe may be designed to capture pre-conversion sequences in the template sequence. Beneficially, such methods may achieve enrichment of targeted regions that is unbiased as to the methylation status estimated in the design of the capture probe. This is advantageous to methods where the nucleic acid population to be enriched, post-conversion and amplification, does not include a copy of the original sequence (pre-conversion) and thus capture probes have to be designed to capture a target region based on an estimated methylation status of the target region, or a given composition of probes have to be designed to capture various degrees of methylation status of the target region.

After hybridization, the hybridized duplex (i.e., the complex that includes the capture probe and amplicon (or converted nucleic acid molecule) can be separated from nucleic acid molecules that do not hybridize to a capture probe.

The method may be used to isolate targeted template sequences from a pool. Thus, in some embodiments, the method may include providing a plurality of nucleic acid molecules, each comprising, in the same strand, a template sequence and a copy sequence, wherein the copy sequence is a copy of the template sequence except that substantially all cytosine bases in the copy sequence are methylated, wherein a first portion of nucleic acid molecules in the plurality of nucleic acid molecules comprises a different template sequence than a second portion of nucleic acid molecules in the plurality of nucleic acid molecules; converting unmethylated cytosine residues in the plurality of nucleic acid molecules to uracil residues, thereby generating a plurality of converted nucleic acid molecules, each converted nucleic acid molecule comprising the copy sequence and a converted template sequence; and hybridizing a plurality of capture probes to at least a portion of the copy sequence. The method may further include amplifying the plurality of converted nucleic acid molecules, thereby substituting uracil residues in the converted template sequence with thymine residues to form a plurality of amplicon, wherein the capture probes hybridize to at least a portion of the copy sequence in the amplicons.

Once separated from non-targeted regions, the nucleic acid molecules may be sequenced as described herein. For example, the nucleic acid molecules may be sequenced to determine a methylation profile of the template sequence.

### Flow Sequencing Methods

Sequencing data can be generated using a flow sequencing method that includes extending a primer bound to a template polynucleotide molecule according to a pre-determined flow cycle where, in any given flow position, a set of nucleotide base types (e.g., 1, 2, or 3 different base types selected from A, C, T and G) is accessible to the extending primer. Fewer base types provided in a given flow provide higher certainty about the precise nucleic acid sequence of the targeted template but provides a smaller sequencing distance per flow. In some embodiments, at least some of the nucleotides of the particular type include a label, which upon incorporation of the labeled nucleotides into the extending primer renders a detectable signal. The resulting sequence by which such nucleotides are incorporated into the extended primer should be the reverse complement of the sequence of the template polynucleotide molecule. In some embodiments, for example, sequencing data is generated using a flow sequencing method that includes extending a primer using labeled nucleotides and detecting the presence or absence of a labeled nucleotide incorporated into the extending primer. Flow sequencing methods may also be referred to as “natural sequencing-by-synthesis,” or “non-terminated sequencing-by-synthesis” methods. Exemplary methods are described in U.S. Pat. No. 8,772,473; International Publication Number WO 2020/227143 A1; and International Publication Number WO 2020/0227137 A1; each of which is incorporated herein by reference in its entirety. While the following description is provided in reference to flow sequencing methods, it is understood that other sequencing methods may be used to sequence all or a portion of the sequenced region.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may be further divided into cycles. Nucleotides are added stepwise, which allows incorporation of the added nucleotide to the end of the sequencing primer of a complementary base in the template strand is present. The cycles may have the same order of nucleotides and number of different base types or a different order of nucleotides and/or a different number of different base types. However, no set of bases (i.e., the one or more different bases simultaneously used in a single flow step) corresponding to a given flow step is repeated in the same cycle as the term is used herein, which can provide as a marker to distinguish between different cycles. Solely by way of example, the order of a first cycle may be A-T-G-C and the order of a second cycle may be A-T-C-G. Further, one or more cycles may omit one or more nucleotides. Solely by way of example, the order of a first cycle may be A-T-G-C and the order of a second cycle may be A-T-C. Alternative orders may be readily contemplated by one skilled in the art. Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E. coli DNA polymerase I, T7 DNA polymerase, bacteriophage T4 DNA polymerase D29 (phi29) DNA polymerase, Taq polymerase, Tth polymerase, TI polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiment, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%. In some implementations, different nucleotide base types may be used in different proportions of labeled to unlabeled nucleotides, e.g., about 60% labeled G, about 50% labeled C, about 50% labeled A, and about 35% labeled T may be used in a particular flow cycle order.

Sequencing data, such as a flowgram, can be generated based on the detection of an incorporated nucleotide and the order of nucleotide introduction. Take, for example, the flowing template sequences: CTG and CAG, and a repeating flow cycle of T-A-C-G (that is, sequential addition of T, A, C, and G nucleotides, which would be incorporated into the primer only if a complementary base is present in the template polynucleotide). A resulting flowgram is shown in Table 1, where 1 indicates incorporation of an introduced nucleotide and 0 indicates no incorporation of an introduced nucleotide. The flowgram can be used to determine the sequence of the template strand.

TABLE 1

| | |
|---|---|
| Cycle 1 | Cycle 2 |

| Sequence | T | A | C | G | T | A | C | G |
|---|---|---|---|---|---|---|---|---|
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

The flowgram may be binary or non-binary. A binary flowgram detects the presence (1) or absence (0) of an incorporated nucleotide. A non-binary flowgram can more quantitatively determine a number of incorporated nucleotide from each stepwise introduction. For example, a sequence of CCG would incorporate two G bases, and any signal emitted by the labeled base would have a greater intensity as the incorporation of a single base. This is shown in Table 1. The non-binary flowgram also indicates the presence or absence of the base but can provide additional information including the number of bases incorporated at the given step.

Prior to generating the sequencing data, the polynucleotide is hybridized to a sequencing primer to generate a hybridized template. The polynucleotide may be ligated to an adapter during sequencing library preparation. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. Pat. No. 10,344,328, which is incorporated herein by reference in its entirety.

The primer hybridized to the polynucleotide is extended through the first region, the second region, and the third region of the polynucleotide. Sequencing data associated with the sequence within the first region and/or the third region may be generated as discussed above. However, the primer is extended through the second region (which is between the first region and the third region) using an accelerated “fast forward” process. That is, extension of the primer through the second region between the first region and the third region of the polynucleotide may proceed faster that the extension of the primer through the first region and/or the third region. For example, extension of the primer through the second region may proceed by extending the primer without detecting the presence or absence of a labeled nucleotide incorporated into the extending primer. During flow sequencing, as discussed above, a labeled nucleotide is incorporated into the extending primer, the hybridized template is washed, and a detector is used to detect a signal from the label of the nucleotide, which indicates whether the nucleotide has been incorporated into the extended primer. However, the detection process takes time, and extension of the primer through the second region can be accelerated by skipping the detection process. In some embodiments, the primer is extended through the second region using unlabeled nucleotides (or using only unlabeled nucleotides), which can further accelerate the rate of primer extension.

Extension of the primer through the second region (for example, a linker between a first portion and first copy portion, or a linker between second portion and a second copy portion) may alternatively or additionally be accelerated by using a mixture of at least two different types of nucleotides in at least one step of the flow order used during extension of the primer through the second region. For example, two different bases, such as G and C, may be used simultaneously in the same step, which extends the primer if a complementary C or G base are present. This accelerates extension of the primer by incorporating consecutive bases into the primer even if those bases are of different base types. In some embodiments, at least one step of the flow order includes 2 different bases. In some embodiments, at least one step of the flow order includes 3 different baes. By way of example, consider a sequence of SEQ ID NO: 1 and the corresponding flow order and flowgram shown in Table 2. The flow order process for extending the sequencing primer hybridized to a polynucleotide containing SEQ ID NO: 1 includes 5 cycles, with Cycles 1, 4, and 5 being the same as each other and Cycles 2 and 3 being the same as each other (with Cycles 1, 4, and 5 being different from Cycles 2 and 3). In this example, each cycle has 4 steps, with Cycles 1, 4, and 5 include the sequential and independent addition of A-C-T-G nucleotides, with a single base type being added at each cycle step. Cycles 2 and 3 include four cycle steps, wherein Step 1 omits A nucleotides (i.e., includes C, T, and G), Step 2 omits, C nucleotides (i.e., includes A, T, and G), Step 3 omits T nucleotides (i.e., includes A, C, and G), and Step 4 omits G nucleotides (i.e., includes A, C, and T). Because Cycles 2 and 3 include multiple different nucleotide base types simultaneously during primer extension, the primer is extended faster than if only a single base type was used at any given step. The flowgram shown in Table 2 for extending the primer against the SEQ ID NO: 1 template using this flow order results in up to 6 bases being added (Cycle 3, Step 3) during the fast forward portion of primer extension. In contrast, Table 3 shows a flowgram of the same SEQ ID NO: 1 using the A-C-T-G cycles with single nucleotides used at each step (similar to Cycles 1, 4, and 5 in Table 2). The flow order used to extend the primer shown in Table 3 requires 10 four-step cycles to extend the primer through the polynucleotide, which is substantially slower than the 5 four-step cycles used to extend the primer through the polynucleotide using the flow order provided in Table 2.

TABLE 2

| Cycle | 1 | | | | 2 | | | | 3 | | | | 4 | | | | 5 | | | |
|---|---|---|---|---|---|---|---|---|---|---|---|---|---|---|---|---|---|---|---|---|
| Cycle Step | 1 | 2 | 3 | 4 | 1 | 2 | 3 | 4 | 1 | 2 | 3 | 4 | 1 | 2 | 3 | 4 | 1 | 2 | 3 | 4 |
| Flow | A | C | T | G | C/T/ | A/T/ | A/C/ | A/C/ | C/T/ | A/T/ | A/C/ | A/C/ | A | C | T | G | A | C | T | G |
| Bases | | | | | G | G | G | T | G | G | G | T | | | | | | | | |
| Number of | 1 | 1 | 1 | 1 | 0 | 2 | 1 | 3 | 4 | 3 | 6 | 2 | 0 | 0 | 0 | 1 | 1 | 1 | 1 | 1 |
| Bases | | | | | | | | | | | | | | | | | | | | |
| Incorporated | | | | | | | | | | | | | | | | | | | | |
| Base(s) | A | C | T | G | — | AA | C | TTA | GGCT | ATA | CGGA | TC | — | — | — | G | A | C | T | G |
| Incorporated | | | | | | | | | | | CG | | | | | | | | | |

Flowgram for SEQ ID NO: 1: 3′-TGACTTGAATCCGATATGCCTGCAGCTGAC-5′

TABLE 3

| Cycle | 1 | | | | 2 | | | | 3 | | | | 4 | | | | 5 | | | |
|---|---|---|---|---|---|---|---|---|---|---|---|---|---|---|---|---|---|---|---|---|
| Cycle Step | 1 | 2 | 3 | 4 | 1 | 2 | 3 | 4 | 1 | 2 | 3 | 4 | 1 | 2 | 3 | 4 | 1 | 2 | 3 | 4 |
| Flow | A | C | T | G | A | C | T | G | A | C | T | G | A | C | T | G | A | C | T | G |
| Bases | | | | | | | | | | | | | | | | | | | | |
| Number of | 1 | 1 | 1 | 1 | 2 | 1 | 2 | 0 | 1 | 0 | 0 | 2 | 0 | 1 | 1 | 0 | 1 | 0 | 1 | 0 |
| Bases | | | | | | | | | | | | | | | | | | | | |
| Incorporated | | | | | | | | | | | | | | | | | | | | |
| Base(s) | A | C | T | G | AA | C | TT | — | A | — | — | CC | — | C | T | — | A | — | T | — |
| Incorporated | | | | | | | | | | | | | | | | | | | | |
| Cycle | 6 | | | | 7 | | | | 8 | | | | 9 | | | | 10 | | | |
| Cycle Step | 1 | 2 | 3 | 4 | 1 | 2 | 3 | 4 | 1 | 2 | 3 | 4 | 1 | 2 | 3 | 4 | 1 | 2 | 3 | 4 |
| Flow | A | C | T | G | A | C | T | G | A | C | T | G | A | C | T | G | A | C | T | G |
| Bases | | | | | | | | | | | | | | | | | | | | |
| Number of | 1 | 1 | 0 | 2 | 1 | 1 | 0 | 1 | 0 | 0 | 1 | 0 | 0 | 1 | 0 | 1 | 1 | 1 | 1 | 1 |
| Bases | | | | | | | | | | | | | | | | | | | | |
| Incorporated | | | | | | | | | | | | | | | | | | | | |
| Base(s) | A | C | — | GG | A | C | — | G | — | — | T | — | — | C | — | G | A | C | T | G |
| Incorporated | | | | | | | | | | | | | | | | | | | | |

Flowgram for SEQ ID NO: 1: 3′-TGACTTGAATCCGATATGCCTGCAGCTGAC-5′

The fast forward method is particularly useful for accelerating primer extension through a region that is not directly sequenced or for which the sequence information is not desired. For example, in reference to Table 2, Cycles 1, 4, and 5 used labeled nucleotides in a stepwise manner to generate sequencing data associated with the first region (Cycle 1) and the third region (Cycles 4 and 5), while the primer was quickly extended through the second region (Cycles 2 and 3) between the first and third region.

Primer extension using flow sequencing allows for long-range sequencing on the order of hundreds or even thousands of bases in length. The number of flow steps or cycles can be increased or decreased to obtain the desired sequencing length. Extension of the primer in the first region or the third region can include one or more flow steps for stepwise extension of the primer using nucleotides having one or more different base types. In some embodiments, extension of the primer in the first region or extension of the primer in the third region includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer in the first region or the third region depends on the sequence of the first region or third region, respectively, and the flow order used to extend the primer in the first region or third region. In some embodiments, the first region or third region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

Primer extension through the second region may proceed through any number of flow steps. In some embodiments, extension of the primer through the second region omits labeled nucleotides, which further increases the feasible extension distance of the primer without polymerase stall. In some embodiments, extension of the primer through the second region includes between 1 and about 10,000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, between about 500 and about 1000 flow steps, between about 1000 flow steps and about 2500 flow steps, between about 2500 flow steps and about 5000 flow steps, or between about 5000 flow steps and about 10,000 flow steps. In some embodiments, extension of the primer through the second region includes more than about 10,000 flow steps. The number of bases incorporated into the primer in the second region depends on the sequence of the second region, and the flow order used to extend the primer in the second region. In some embodiments, the second region is about 1 base to about 50,000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, about 2000 bases to about 2500 bases in length, about 2500 to about 5000 bases in length, about 5000 to about 10,000 bases in length, about 10,000 to about 25,000 bases in length, or about 25,000 to about 50,000 bases in length. In some embodiments, the length of the second region is more than about 50,000 bases in length.

Extension of the primer can proceed through the first region, the second region, and the third region, wherein the primer is extended through the first region and the third region using labeled nucleotides. Detection of nucleotides incorporated into the extending primer can be detected to generate sequencing data. Extension of the primer through the second region can occur at a faster rate than extension of the primer through the first and/or third regions, for example without detecting the presence or absence of a label of a nucleotide incorporated into the extending primer, or by including a mixture of at least two different types of nucleotide bases to extend the primer (wherein the extension of the primer through the first and/or third relies on fewer different types of nucleotide bases.

The fast forward process may be used to extend the sequencing primer through the linker sequence between the template portion and the copy portion of the nucleic acid construct, either with or without conversion of the methylated cytosine bases (or non-methylated cytosine bases) to uracil. For example, a method for sequencing may include (a) providing a nucleic acid molecule comprising, in order, a first sequence, a second sequence, and a third sequence, wherein the first sequence is a copy of the third sequence except that (1) at least one base corresponding to a cytosine base in the third sequence is a thymine in the first sequence, or (2) at least one base corresponding to a guanine base in the third sequence is an adenine in the first sequence; (b) sequencing the first sequence by, for each of a plurality of first flow steps in a plurality of first flow cycles, (i) providing labeled nucleotides of a single base type to a primer hybridized to the nucleic acid molecule, and (ii) detecting one or more signals indicative of incorporation, or lack thereof, of a labeled nucleotide in the primer; (c) processing the second sequence by, for at least one of a plurality of second flow steps in a plurality of second flow cycles, providing nucleotides of two or three base types to the primer; and (d) sequencing the third sequence by, for each of a plurality of third flow steps in a plurality of third flow cycles, (i) providing labeled nucleotides of a single base type to the primer, and (ii) detecting one or more signals indicative of incorporation, or lack thereof, of a labeled nucleotide in the primer.

The fast forward process may also be applied for generating sequencing data. For example, the sequencing data generation may include providing three different base types in one flow and the additional base in the following flow, in a repeated pattern. The copy may be sequenced using a different set of three base types followed by sequencing using the fourth base type. By way of example, one copy of the template may be sequenced by iteratively providing (1) a sequencing flow comprising A, C, and T bases and detecting incorporation of a labeled base, and (2) a sequencing flow comprising G base and detecting incorporation of a labeled base. The second copy of the template may be sequenced using a different combination, for example, (1) a sequencing flow comprising A, T, and G bases and detecting incorporation of a labeled base, and (2) a sequencing flow comprising C base and detecting incorporation of a labeled base.

For example, a method for sequencing can include (a) providing a nucleic acid molecule comprising a first sequence and a second sequence, wherein the first sequence and the second sequence are identical; (b) sequencing the first sequence by, for each cycle of a plurality of first flow cycles, (i) providing labeled nucleotides of a first combination of three base types to a primer hybridized to the nucleic acid molecule, (ii) detecting one or more signals indicative of incorporation, or lack thereof, of one or more labeled nucleotides of the first combination of three base types in the primer, and (iii) providing nucleotides of a fourth base type different from the three base types in the first combination; and (c) sequencing the second sequence by, for each cycle of a plurality of second flow cycles, (i) providing labeled nucleotides of a second combination of three base types to the primer, wherein the second combination is different from the first combination, (ii) detecting one or more signals indicative of incorporation, or lack thereof, of one or more labeled nucleotides of the second combination of three base types in the primer, and (iii) providing nucleotides of a fifth base type different from the three base types in the second combination. In some implementations, nucleotides of the fourth base type provided in step (b) are labeled, and step (b) further includes (iv) detecting one or more additional signals indicative of incorporation, or lack thereof, of one or more additional labeled nucleotides of the fourth base type. In some implementations, nucleotides of the fifth base type provided in step (c) are labeled, and step (c) further comprises (iv) detecting one or more additional signals indicative of incorporation, or lack thereof, of one or more additional labeled nucleotides of the fifth base type. In some implementations, the method further includes comparing, or combining, first sequencing data corresponding to the one or more signals detected in step (b) and second sequencing data corresponding to the one or more signals detected in step (c), to determine at least a portion of the first sequence.

### Methylation Status Data

As discussed above, the nucleic acid sequence of the first copy portion or the second copy portion is not altered by conversion of the methylated or non-methylated cytosine to uracil (and subsequently, after amplification, thymine). Thus, the first copy portion and the second copy portion may be used to generate sequencing data indicating the nucleic acid sequence of the first template sequence and the second template sequence, respectively. However, this sequencing data alone does not reflect the methylation status of the cytosine bases in the first and second template sequences. Methylation status data (i.e., sequencing data, which may be obtained using a sequencing process designed rapidly extend the primer through a target region while obtaining information about the methylation status of cytosine in the target region) may be obtained from the first portion and/or the second portion (i.e., the portion of the construct corresponding to the first template sequence and/or the second template sequence). Differences between the sequencing data corresponding sequencing data obtained from the first/second copy portion and the first/second portion are indicative of the methylation states in the first/second template sequence.

The sequencing data for the first copy portion and the sequencing/methylation status data for the first portion may be obtained from the same first strand sequencing read. That is, a single sequencing primer (for example, hybridized to a hybridization sequence in a sequencing adapter) may be extended through the first copy portion to obtain sequencing data for the first copy portion, through the first linker region (which may be through a fast forward process, wherein sequencing data need not be collected for the linker region), and the first portion to obtain sequencing/methylation status data for the first portion. A similar process may be applied to the second strand.

The methylation profiling data of a template sequence may include the location of methylated cytosine or non-methylated cytosine in the template sequence. That is, the sequence of the first or second copy portion can be taken as the ground truth for the sequence of the respective sequence. A thymine base in the sequence of the first (or second) portion that corresponds to a cytosine base in the first (or second) copy portion indicates a conversion of a non-methylated cytosine originally found in the first (or second) template if non-methylated cytosine bases were converted to uracil in the conversion reaction. Alternatively, a thymine base in the in the sequence of the first (or second) portion that corresponds to a cytosine base in the first (or second) copy portion indicates a conversion of a methylated cytosine originally found in the first (or second) template if methylated cytosine bases were converted to uracil in the conversion reaction. Thus, the methylation profiling data can include a location of methylated cytosine or non-methylated cytosine in the first template sequence or the second template sequence.

In some implementations, the methylation profiling data of a template sequence may include a density or signal intensity of methylated cytosine (or non-methylated cytosine) in the first or second template sequence. That is, it may not be necessary to know the precise locations of the methylated or non-methylated cytosine within the template sequence, but it is sufficient to know what proportion of cytosine bases in the template sequence are methylated. Thus, the first portion or the second portion may be assayed (e.g., by a sequencing process) after conversion to detect signals indicating a conversion of a methylated cytosine to a thymine (or non-methylated cytosine to a thymine).

As discussed above, the sequencing data for determining a nucleic acid sequence (e.g., of a first copy portion or a second copy portion) can include, for each of a plurality of sequencing flow steps, (i) extending the sequencing primer by providing, to the hybridized template, labeled nucleotides of a single base type, and (ii) detecting a signal indicating incorporation of a labeled nucleotide into the extending sequencing primer. While providing nucleotides of a single base type in any given flow step provides accurate sequencing information, the process is relatively slow. Since the precise nucleic acid sequence of the first portion or second portion is not always necessary, described herein is a process for quickly generating methylation status data.

Methylation status data may be generated from the first template portion or the second template portion by, iteratively, (i) extending the sequencing primer by providing, to the hybridized template, a mixture of thymine, cytosine, and adenine nucleotides, (ii) extending the sequencing primer by providing, to the hybridized template, a mixture of cytosine and guanine bases, wherein at least a portion of the cytosine bases are labeled, and (iii) detecting a signal indicating incorporation of a labeled cytosine base into the extending sequencing primer. The mixture of thymine, cytosine, and adenine nucleotides allows primer extension until a cytosine is present in the template strand. That is, the thymine, cytosine, and adenine bases can base pair with any thymine, guanine, or adenine base in the template, but stalls where a cytosine base is present in the template. When non-methylated cytosine bases are converted to uracil (and subsequently adenine), primer extension does not stall at loci where the original template had a non-methylated cytosine; instead, the primer extension only stalls when the original template had a methylated cytosine. Similarly, when methylated cytosine bases are converted to uracil (and subsequently adenine), primer extension does not stall when the original template had a methylated cytosine; instead, the primer extension only stalls when the original template had a non-methylated cytosine.

Methylated cytosine bases most frequently occur within CpG sites. Thus, a single cytosine (i.e., not flanked by a cytosine) in the template is considered unlikely to be methylated in the original template sequence, although may be residual from incomplete conversion (e.g., the non-methylated cytosine was not converted to uracil because the reaction did not go to completion). By labeling the cytosine bases (rather than guanine bases), no detectable signal is produced due to an isolated cytosine. But including a mixture of cytosine and guanine bases, wherein at least a portion of the cytosine bases are labeled, CpG sites, wherein the cytosine base remains unconverted, will provide a detectable signal from incorporation of the labeled cytosine nucleotide resulting from the G in the template strand.

The sequencing data and the methylation status data may be obtained from a single sequencing read. For example, a sequencing primer may be hybridized to an adapter sequence attached to a nucleic acid strand that includes a converted template portion and a converted copy portion, wherein the converted template portion and the converted copy portion differ based on the methylation profile of the original template sequence. The primer is extended through the converted copy portion, generating sequencing data from the converted copy portion, and then continues to extend through the converted template portion, generating methylation status data from the converted template portion. Flow sequencing methods described herein (which may be specifically designed to generate the methylation status data, as discussed) can therefore be used to generate both the sequencing data indicating a nucleic acid sequence and the methylation status data in a single read.

The converted template portion and the converted copy portion may be separated by a linker. The sequencing primer may be extended through the linker using a “fast forward” extension process, for example by including flows that include two or three different nucleotide base type in a flow step, or by omitting a detection step (or both). As discussed above, the linker may have a known sequence. Thus, in some embodiments, the plurality of flow steps used to extend the sequencing primer through the linker ma may be pre-determined (e.g., optimized) based on the known sequence. That is, for each of a plurality of extension flow steps, a mixture of two or three different base types may be provided to the hybridized template, wherein the two or three different base types provided to the hybridized template are selected based on a known sequence of the nucleic acid linker.

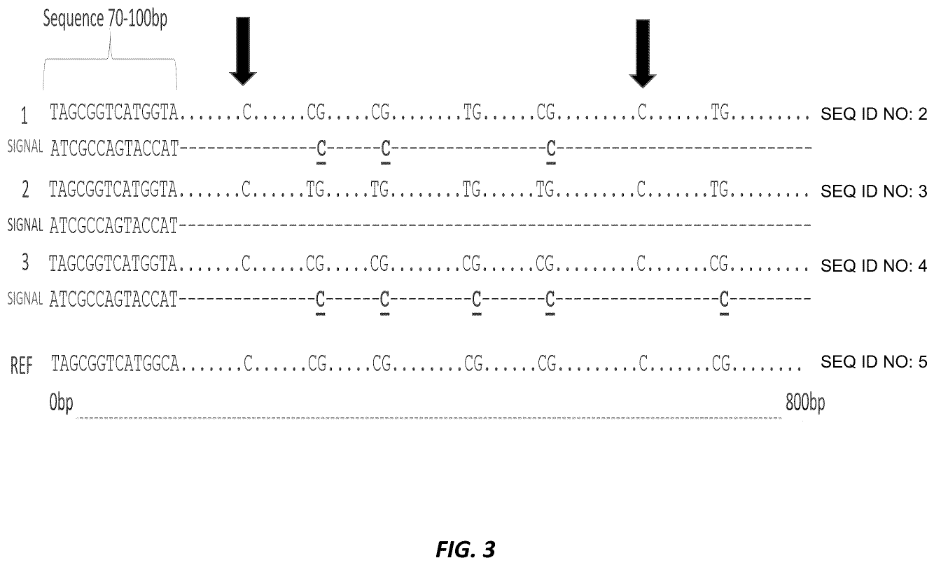

Methylation profiling data generation need not depend on knowing the sequencing data for a particular nucleic acid sequence (e.g., the entirety of a particular nucleic acid sequence does not need to be sequenced in order to determine a number or proportion of methylated/unmethylated CpG sites). For example, as discussed herein, in some implementations, it is sufficient to know the methylation density of a nucleic acid sequence. The methylation status data generation method described herein can provide such information. For example, non-methylated cytosine bases in a nucleic acid molecule may converted to uracil (or, alternatively, methylated cytosine bases in the nucleic acid molecule converted to uracil) to generate a converted nucleic acid molecule. The converted nucleic acid molecule may be amplified (for example, by PCR application), thereby converting the uracil bases to thymine bases in the resulting amplified converted nucleic acid molecules. The amplified nucleic acid molecules can include a sequencing adapter, which includes a hybridization site that hybridizes to a primer. Primers can then be hybridized to the amplified nucleic acid molecules to form hybridized templates. The primer can then be extended through at least a portion of the nucleic acid molecule to generate methylation status data. For example, generating the methylation status data can include, iteratively, (i) extending the primers by providing, to the hybridized templates, a mixture of thymine, cytosine, and adenine nucleotides, (ii) extending the sequencing primer by providing, to the hybridized templates, a mixture of cytosine and guanine bases, wherein at least a portion of the cytosine bases are labeled, and (iii) detecting a signal indicating incorporation of a labeled cytosine base into the extending sequencing primer. Optionally, sequencing data indicative of a nucleic acid sequence of a second portion of the nucleic acid molecule may be generated. Knowing the sequence of a portion of the nucleic acid molecule (either upstream or downstream of the portion of the nucleic acid molecule used to generate the methylation status data) can be used to identify a genomic locus for the methylation status. For example, the sequence of the second portion of the nucleic acid molecule may be mapped (e.g., aligned) to a reference sequence for the genome to identify a genomic locus of the nucleic acid molecule. Since the methylation status data is generated from a portion of the nucleic acid molecule (i.e., the first portion) proximal to the portion of the nucleic acid molecule used to generate the sequencing data (i.e., the second portion), and the locus of the mapped sequence within the genome indicates the locus of the methylation status data. In some implementations, as illustrated in FIG. 3, the sequencing data is generated prior to generating the methylation status data, as signal to noise may decrease as the primer is extended and a clear signal is needed to determine the sequence of the nucleic acid molecule than the methylation status data.

FIG. 3 illustrates exemplary methylation status data that may be obtained using the method described herein. The illustrated example shows three identical nucleic acid sequences aligned with a reference sequence, where the nucleic acids differ in methylation profile. Below each sequence is the respective signal that may be detected by flowing a complementary labeled nucleotide in a flow sequencing process. The first 70-100 bases of the nucleic acid molecule are sequenced using the standard flow sequencing process, wherein a single base type is provided in each sequencing flow, according to a sequencing flow cycle. The methylation status data for each sequence is then collected by iteratively, (i) extending the sequencing primers by providing, to the hybridized templates, a mixture of thymine, cytosine, and adenine nucleotides, (ii) extending the sequencing primers by providing, to the hybridized templates, a mixture of cytosine and guanine bases, wherein at least a portion of the cytosine bases are labeled, and (iii) detecting a signal indicating incorporation of a labeled cytosine base into the extending sequencing primer. Sequence 2 assumes no methylated cytosine in the original template. Thus, substantially all of the cytosine bases in the original template are converted to thymine bases. However, if the conversion reaction is incomplete, residual cytosine bases may remain the converted nucleic acid molecule, as indicated by the arrows. When the mixture of thymine, cytosine, and adenine bases are provided to extend the sequencing primer, the primer stalls at the residual cytosine. However, no signal is produced because there is no complementary guanine base to allow incorporation of a labeled cytosine within a mixture of guanine and labeled cytosine bases are provided. Because this cytosine is not within a CpG site, it is unlikely that this cytosine was a methylated cytosine in the original template; thus, the no-signal result avoids a false positive. Non-methylated cytosine bases in sequence 2 within CpG sites are converted to thymine residues, and the mixture of thymine, cytosine, and adenine bases causes the primer to extend through these bases. Thus, sequence 2 produces no methylation signal. Sequence 3 assumes all cytosine bases within CpG sites are methylated. When the sequence extends to a cytosine in the template strand, primer extension stalls with the mixture of thymine, cytosine, and adenine bases (i.e., excluding guanine) bases is provided. When the primer extends to a cytosine base adjacent to a guanine base, a signal can be detected after a mixture of guanine and labeled cytosine is provided to extend the primer. Sequence 1 assumes some cytosine bases in CpG sites are methylated and some cytosine bases in CpG sites are non-methylated, thus providing a signal at CpG sites with a methylated cytosine base in the original template strand.

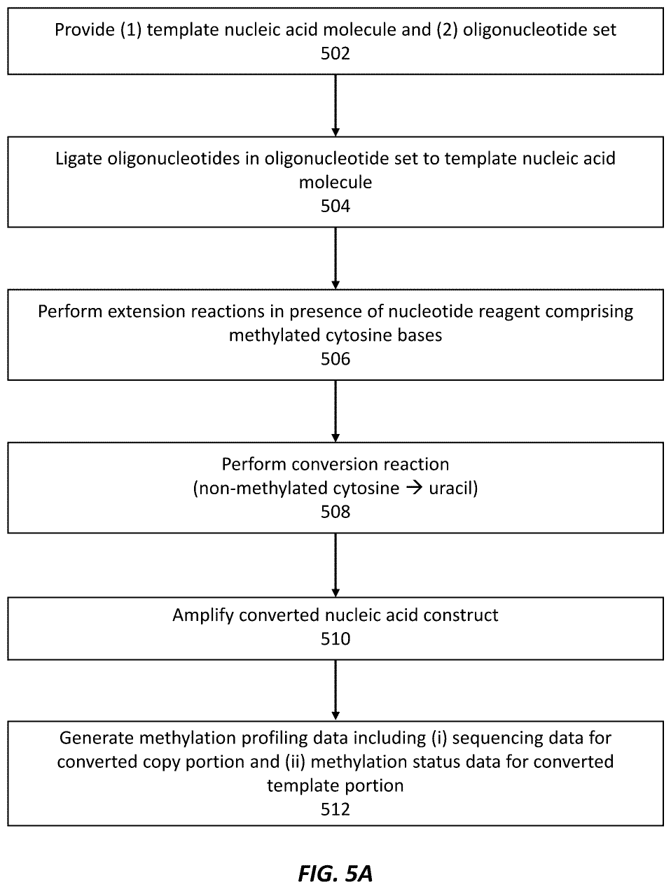

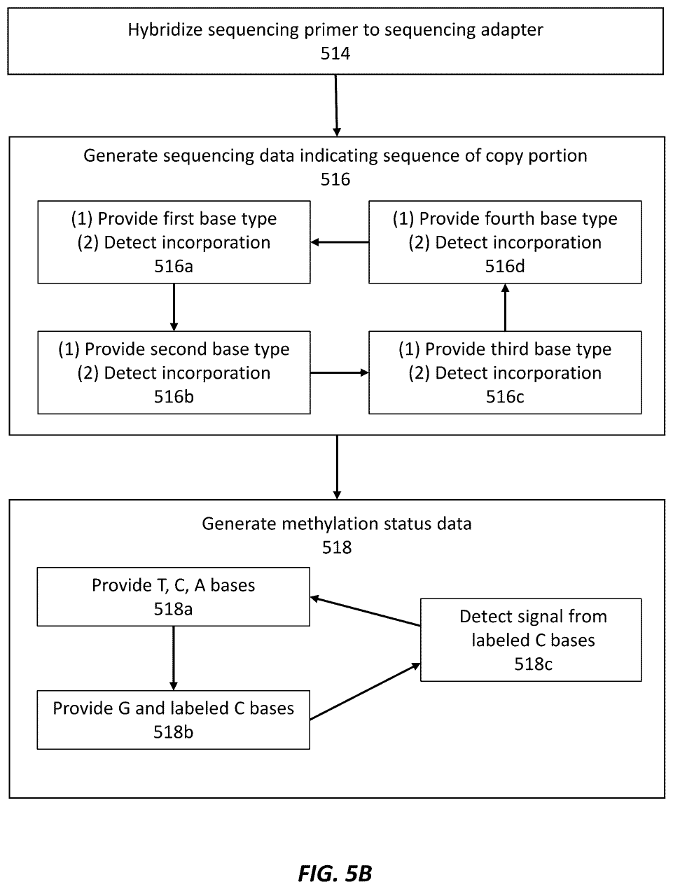

FIG. 5A illustrates an exemplary method for obtaining methylation profiling data for a nucleic acid molecule. At 502, a template nucleic acid molecule and an oligonucleotide set are provided. The template nucleic acid molecule is a duplex molecule with a “top” strand and a “bottom” strand. The oligonucleotide set includes four oligonucleotides, portions of which hybridize (e.g., through reverse complementarity) to form a complex comprising the four-oligonucleotides. The oligonucleotide set can assemble such that a 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide. The first oligonucleotide may further include a 5′ portion that include adapter sequence (e.g., includes a hybridization site for a sequencing primer). Similarly, the second oligonucleotide may include a 5′ portion that include adapter sequence (e.g., includes a hybridization site for a sequencing primer), which may be the same or different as the adapter sequence included in the first oligonucleotide.

The oligonucleotide set is then ligated to the template nucleic acid at 504. For example, a 3′ terminus of the first oligonucleotide can be ligated to a 5′ terminus of the first strand of the template nucleic acid, a 5′ terminus of the second oligonucleotide can be ligated to a 3′ terminus of the second strand, a 5′ terminus of the third oligonucleotide can be ligated to a 3′ terminus of the first strand, and a 3′ terminus of the fourth oligonucleotide can be ligated to a 5′ terminus of the second strand.

At 506, extension reactions are performed in the presence of a nucleotide reagent comprising methylated cytosine bases. Substantially all cytosine bases in the nucleotide reagent may be methylated cytosine bases. The nucleotide regent also includes other nucleotides necessary for the extension reaction (e.g., A, T, and G bases). The resulting nucleic acid construct includes a first strand comprising the first template sequence portion (“original top”) and a first copy portion (“copied top”), and a second strand comprising the second template sequence portion (“original bottom”) and a second copy portion (“copied bottom”).

At 508 the construct subjected to a conversion reaction, which converts non-methylated cytosine to uracil, thereby forming a converted nucleic acid construct. The converted nucleic acid construct is amplified at 510, which replaces uracil bases with thymine bases in the amplified product.

At 512, methylation profiling data is generated, which includes sequencing data obtained from the converted copy portion and methylation status data from the converted template portion. FIG. 5B provides further detail for obtaining methylation profiling data in accordance with some embodiments. At 514, a sequencing primer is hybridized to a sequencing adapter of a converted strand of the converted nucleic acid molecule. At 516, sequencing data is generated from the converted copy portion. The sequencing data is generated using a plurality of sequencing flow steps in a flow cycle order. The primer is extended as the sequencing data is generated. In each flow step, labeled nucleotides of a single base type are provide to the hybridized template, following by detecting a signal indicating incorporation of a labeled nucleotide into the extending sequencing primer. At 518, methylation status data is generated for the converted template portion. The sequencing primer is further extended as the methylation status data is generated. A mixture of thymine, cytosine and adenine bases pare provided to the hybridized template at 518a, and the primer stalls when a cytosine base is present in the template strand. Guanine and cytosine bases, wherein at least a portion of the cytosine bases are labeled, are then provided at 518b. At 518c, incorporation of labeled C bases is detected, which indicates a methylated cytosine in the original template.

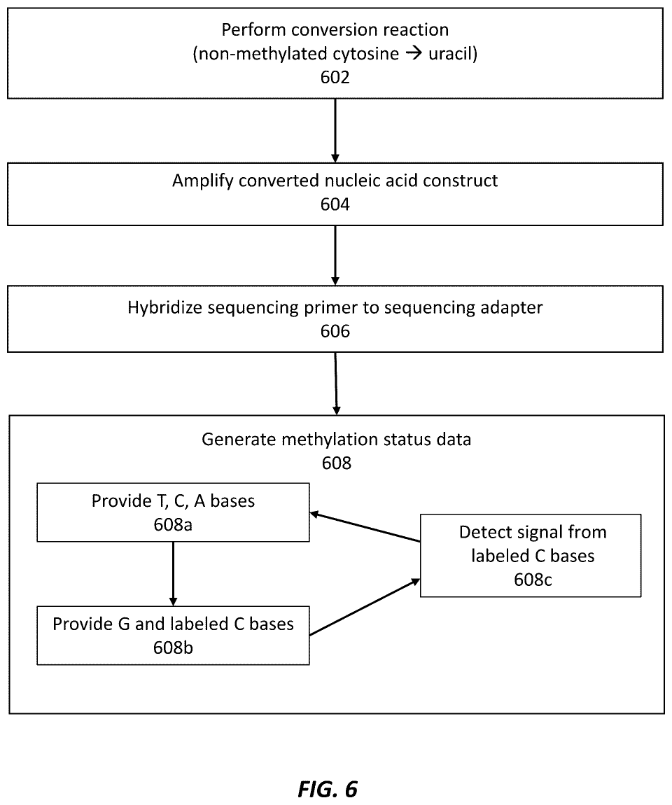

FIG. 6 illustrates a method of generating methylation status data for a target nucleic acid molecule. At 602, non-methylated cytosine bases are converted to uracil bases (or methylated cytosine bases are converted to uracil bases) in a target nucleic acid molecule, thereby generating a converted nucleic acid molecule. At 604, the converted nucleic acid molecule is amplified, thereby converted the uracil bases to thymine bases. At 606, a sequencing primer is hybridized to the converted nucleic acid molecule, for example at a hybridization site within a sequencing adapter attached to the target nucleic acid molecule. At 608, methylation status data is generated. The primer is extended as the methylation status data is generated. A mixture of thymine, cytosine and adenine bases pare provided to the hybridized template at 608a, and the primer stalls when a cytosine base is present in the template strand. Guanine and cytosine bases, wherein at least a portion of the cytosine bases are labeled, are then provided at 608b. At 608c, incorporation of labeled C bases is detected, which indicates a methylated cytosine in the original template.

### Repeat Sequencing for Variant Detection

Sensitivity of a short genetic variant detected depends on the flow cycle order used to sequencing the nucleic acid molecule. Thus, a template sequence may be sequenced using two or more different flow cycle orders. A variant missed using the first flow cycle order may be detected using the second flow cycle order. The nucleic acid construct described herein (e.g., without converting methylated or non-methylated cytosine bases) provides two identical copies of the template nucleic acid molecule, which allows for convenient re-sequencing of the template sequence using a different flow cycle order.

As discussed above, the nucleic acid construct may be synthesized by providing a template nucleic acid molecule and an oligonucleotide set of four oligonucleotides. The template nucleic acid may be, for example, the duplex nucleic acid molecule obtained from the biological sample form a subject. The template nucleic acid molecule includes a first strand comprising a first template sequence (i.e., corresponding to the first portion in the construct discussed above) and a second strand comprising a second template sequence (corresponding to the second portion in the construct discussed above). The template nucleic acid may be prepared for construct synthesis, for example by nucleic acid end repair and/or A-tailing. The first strand and/or second strand of the nucleic acid molecule may be a cfDNA molecule.

The oligonucleotide set includes four oligonucleotides, portions of which hybridize (e.g., through reverse complementarity) to form a complex comprising the four-oligonucleotides. The oligonucleotide set can assemble such that a 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide. The first oligonucleotide may further include a 5′ portion that include adapter sequence (e.g., includes a hybridization site for a sequencing primer). Similarly, the second oligonucleotide may include a 5′ portion that include adapter sequence (e.g., includes a hybridization site for a sequencing primer), which may be the same or different as the adapter sequence included in the first oligonucleotide.

Optionally, the second oligonucleotide is cross-linked to the third oligonucleotide through a crosslinker, which may be a reversible crosslinker. Exemplary reversible crosslinkers include a psoralen crosslinker or a 3-cyanovinylcarbazole (CNVK) crosslinker. Other reversible crosslinkers are known in the art. The crosslinker can crosslink the portion of the second oligonucleotide that hybridizes to the portion of the third oligonucleotide. For example, the 3′ portion of the second oligonucleotide can include a first member of a crosslinker (e.g., a reversible crosslinker) and the 3′ portion of the third oligonucleotide can include a second member of the crosslinker.

The oligonucleotide set is then ligated to the template nucleic acid. For example, a 3′ terminus of the first oligonucleotide can be ligated to a 5′ terminus of the first strand of the template nucleic acid, a 5′ terminus of the second oligonucleotide can be ligated to a 3′ terminus of the second strand, a 5′ terminus of the third oligonucleotide can be ligated to a 3′ terminus of the first strand, and a 3′ terminus of the fourth oligonucleotide can be ligated to a 5′ terminus of the second strand. In some implementations, the second oligonucleotide is cross-linked to the third oligonucleotide prior to the ligating. In some implementations, the second oligonucleotide is cross-linked to the third oligonucleotide after the ligating. The resulting nucleic acid construct is a partially circular nucleic acid molecule that includes a first strand comprising a first template sequence and a second strand comprising a second template sequence, wherein the first template sequence is a reverse complement of the second template sequence.

An extension reaction is then performed on the partially circular nucleic acid molecule. The 3′ terminus of the second oligonucleotide is extended using, in order, a portion of the third oligonucleotide, the first strand and the first oligonucleotide as a template. The 3′ terminus of the third oligonucleotide is also extended using, in order, a portion of the second oligonucleotide, the second strand, and the fourth oligonucleotide as a template. In some implementations, the optional reversible crosslinker is reversed after the extension reactions. The resulting nucleic acid molecule construct includes a first strand comprising the first template sequence portion and a first copy portion, and a second strand comprising the second template sequence portion and a second copy portion.

The first strand and/or second strand of the construct may then be sequence using different flow orders for the template sequence portion and the corresponding copy portion. For example, a sequencing primer can be hybridized to the first or second strand to form a hybridized template. First sequencing data can be generated for the copy portion by, for each of a plurality of sequencing flow steps according to a first flow order, (i) extending the sequencing primer by providing, to the hybridized template, labeled nucleotides of a single base type, and (ii) detecting a signal indicating incorporation of a labeled nucleotide into the extending sequencing primer. Second sequencing data can also be generated for the template sequence portion by, for each of a plurality of sequencing flow steps according to a second flow order, (i) extending the sequencing primer by providing, to the hybridized template, labeled nucleotides of a single base type, and (ii) detecting a signal indicating incorporation of a labeled nucleotide into the extending sequencing primer. The first flow order and the second flow order are different so that the resulting sequencing data is different. Different flow orders can result in different sensitivities for different contextual variants.

The template sequence portion and the corresponding copy portion may be separated by a nucleic acid linker. The sequence of the nucleic acid linker may be known apriori or may not be of particular interest. Thus, the sequencing primer can be extended through the linker sequence using a “fast forward” process. For example, A nucleic acid molecule may be sequenced by (a) providing a nucleic acid molecule comprising, in order, a first sequence (e.g., a copy portion), a second sequence (e.g., a linker sequence), and a third sequence (e.g., a template sequence portion), wherein the first sequence and the third sequence are identical; (b) sequencing the first sequence by, for each of a plurality of first flow steps in a plurality of first flow cycles, (i) providing labeled nucleotides of a single base type to a primer hybridized to the nucleic acid molecule, and (ii) detecting one or more signals indicative of incorporation, or lack thereof, of a labeled nucleotide in the primer; (c) processing the second sequence by, for at least one of a plurality of second flow steps in a plurality of second flow cycles, providing nucleotides of two or three base types to the primer; and (d) sequencing the third sequence by, for each of a plurality of third flow steps in a plurality of third flow cycles, (i) providing labeled nucleotides of a single base type to the primer, and (ii) detecting one or more signals indicative of incorporation, or lack thereof, of a labeled nucleotide in the primer.

### Mutagenesis Sequencing

The nucleic acid molecule construct having two copies of the template sequence may be constructed in the presence of a mutagenesis agent, which can introduce random mutations into the copy portion(s) of the first or second strand. Random mutations will lead to breakage of long homopolymer regions, which are frequently difficult to sequence using standard flow sequencing methods. Exemplary mutagenesis agents include, but are not limited to, 8-Oxo-dGTP, dPTP, 8-oxo-dG (8-oxo-2′-deoxyguanosine), 5Br-dUTP, 20H-dATP, and dITP. The mutagenesis agent may introduce on or more mutations into the copy portion, for example one or more of A:T to C:G, T:A to G:C, A:T to T:A, A:T to G:C, G:C to A:T, T:A to C:G, and G:C to T:A.

Thus, the method of forming the construct may include performing extension reactions, in the presence of a mutagenesis agent, on a partially circular nucleic acid molecule comprising a first strand comprising a first template sequence and a second strand comprising a second template sequence, wherein the first template sequence is a reverse complement of the second template sequence, thereby generating a nucleic acid molecule comprising a first strand comprising the first template sequence and a first copy portion, wherein the first copy portion is a copy of the first template sequence except that at least 1 base is different due to mutagenesis; and a second strand comprising the second template sequence and a second copy portion, wherein the second copy portion is a copy of the second template sequence except that at least 1 base is different due to mutagenesis. In some implementations, the first copy portion (or second copy portion) is a copy of the first template sequence (or second template sequence) except that at least 5 bases (or at least 10 bases) are different as a result of the mutagenesis agent. The nucleic acid construct may further be amplified (for example using PCR amplification).

The first template sequence and the first copy portion (or the second template sequence and the second copy portion) may be sequenced, for example using the flow sequencing methods described herein.

Data indicative of the length of a homopolymer sequence in the first template sequence may be determined based at least in part on processing two or more of first sequencing data corresponding to the first template sequence, second sequencing data corresponding to the first copy portion, third sequencing data corresponding to the second template sequence, and fourth sequencing data corresponding to the second copy portion.

### Pseudo Paired End Sequencing

A nucleic acid construct that includes a template portion and a cop portion in the first and second strands may be synthesized (e.g., via extension reactions) in the presence of deoxyuridine (e.g., up to about 1%, up to about 2%, up to about 3%, up to about 5%, up to about 7%, or up to about 10% of all nucleotides in the synthesis reaction). The resulting nucleic acid construct may be subjected to a cleavage reaction at one or more deoxyuridine sites (for example using a uracil-specific excision reagent, such one or both of a uracil DNA glycosylase (UDG) and an endonuclease (e.g., Endonuclease VIIII), for example a USER® Enzyme (New England BioLabs)) to generate a truncated molecule. A single stranded DNA portion of the truncated molecule may be digested, for example with an exonuclease, to generate a second truncated molecule. One or more sequencing adapters may be coupled to the second truncated molecule.

By way of example, a method may include performing extension reactions, in the presence of deoxyuridine at a concentration of up to 10% of all nucleotides, on a partially circular nucleic acid molecule comprising a first strand comprising a first template sequence and a second strand comprising a second template sequence, wherein the first template sequence is a reverse complement of the second template sequence, thereby generating a nucleic acid molecule comprising a first strand comprising the first template sequence and a first copy portion, wherein the first copy portion is a copy of the first template sequence except that at least base corresponding to a thymine in the first template sequence is a deoxyuridine; and a second strand comprising the second template sequence and a second copy portion, wherein the second copy portion is a copy of the second template sequence except that at least base corresponding to a thymine in the second template sequence is a deoxyuridine.

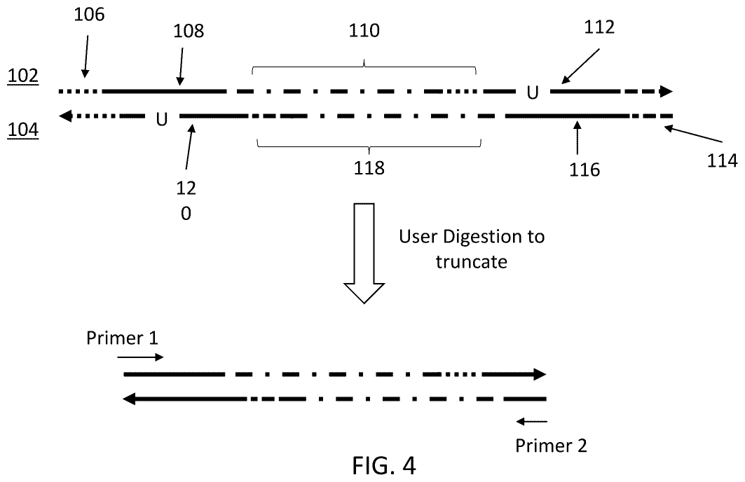

FIG. 4 illustrates an exemplary method of making a construct for pseudo paired end sequencing.

1.-101. (canceled)

102. A method, comprising:
(a) providing:
a template nucleic acid molecule comprising a first strand comprising a first template sequence and a second strand comprising a second template sequence, wherein the first template sequence is a reverse complement of the second template sequence; and
an oligonucleotide set, comprising a first oligonucleotide, a second oligonucleotide, a third oligonucleotide, and a fourth oligonucleotide, wherein:
a 3′ portion of the first oligonucleotide hybridizes to a 5′ portion of the second oligonucleotide,
a 3′ portion of the second oligonucleotide hybridizes to a 3′ portion of the third oligonucleotide, and
a 5′ portion of the third oligonucleotide hybridizes to a 3′ portion of the fourth oligonucleotide;
(b) ligating:
a 3′ terminus of the first oligonucleotide to a 5′ terminus of the first strand,
a 5′ terminus of the second oligonucleotide to a 3′ terminus of the second strand,
a 5′ terminus of the third oligonucleotide to a 3′ terminus of the first strand, and
a 3′ terminus of the fourth oligonucleotide to a 5′ terminus of the second strand;
(c) performing extension reactions in the presence of a nucleotide reagent comprising methylated cytosine bases, comprising:
extending a 3′ terminus of the second oligonucleotide using, in order, a portion of the third oligonucleotide, the first strand, and the first oligonucleotide as a template,
extending a 3′ terminus of the third oligonucleotide using, in order, a portion of the second oligonucleotide, the second strand, and the fourth oligonucleotide as a template,
thereby generating a nucleic acid molecule comprising:
a first strand comprising the first template sequence and a first copy portion, and a second strand comprising the second template sequence and a second copy portion;
(d) sequencing the first strand, comprising:
hybridizing a sequencing primer to the first strand to form a hybridized template;
generating first sequencing data for the first copy portion, comprising extending the sequencing primer by, for each of a plurality of sequencing flow steps according to a first flow order, (i) providing, to the hybridized template, labeled nucleotides of a single base type, and (ii) detecting a signal indicating incorporation, or lack thereof, of a labeled nucleotide into the extending sequencing primer; and
generating second sequencing data for the first template sequence, comprising extending the sequencing primer by, for each of a plurality of sequencing flow steps according to a second flow order, (i) providing, to the hybridized template, labeled nucleotides of a single base type, and (ii) detecting a signal indicating incorporation, or lack thereof, of another labeled nucleotide into the extending sequencing primer,
wherein the first flow order and the second flow order are different.

103. The method of claim 102, wherein the labeled nucleotides are non-terminated.

104. The method of claim 102, further comprising, prior to sequencing of (d), converting a non-methylated cytosine in the first strand to a uracil to generate a converted nucleic acid molecule comprising a converted first copy portion and a converted first template sequence.

105. The method of claim 104, further comprising comparing the first sequencing data and the second sequencing data, wherein one or more differences between the first sequencing data and the second sequencing data are indicative of methylation status in the first template sequence.

106. The method of claim 102, further comprising comparing, or combining, the first sequencing data and the second sequencing data to determine a sequence of at least a portion of the first template sequence.

107. The method of claim 106, wherein one or more differences between the first sequencing data and the second sequencing data are indicative of variants in the first template sequence.

108. The method of claim 102, wherein providing, to the hybridized template, labeled nucleotides of a single base type in sequencing flow steps of the first flow order or the second flow order further comprises providing unlabeled nucleotides of the single base type.

109. The method of claim 102, further comprising sequencing the second strand, comprising:
hybridizing a second sequencing primer to the second strand to form a second hybridized template;
generating third sequencing data for the second template sequence, comprising extending the second sequencing primer by, for each of a plurality of sequencing flow steps according to a third flow order, (i) providing, to the second hybridized template, labeled nucleotides of a single base type, and (ii) detecting a signal indicating incorporation, or lack thereof, of a labeled nucleotide into the second extending sequencing primer; and
generating fourth sequencing data for the second copy portion, comprising extending the second sequencing primer by, for each of a plurality of sequencing flow steps according to a fourth flow order, (i) providing, to the second hybridized template, labeled nucleotides of a single base type, and (ii) detecting a signal indicating incorporation, or lack thereof, of another labeled nucleotide into the second extending sequencing primer,
wherein the third flow order and the fourth flow order are different.

110. A method for sequencing, comprising:
(a) providing a nucleic acid molecule comprising, in order, a first sequence, a second sequence, and a third sequence, wherein the first sequence and the third sequence are identical;
(b) sequencing the first sequence by, for each of a plurality of first flow steps in a plurality of first flow cycles, (i) providing labeled nucleotides of a single base type to a primer hybridized to the nucleic acid molecule, and (ii) detecting one or more signals indicative of incorporation, or lack thereof, of a labeled nucleotide in the primer;
(c) processing the second sequence by, for at least one of a plurality of second flow steps in a plurality of second flow cycles, providing nucleotides of two or three base types to the primer; and
(d) sequencing the third sequence by, for each of a plurality of third flow steps in a plurality of third flow cycles, (i) providing other labeled nucleotides of another single base type to the primer, and (ii) detecting one or more signals indicative of incorporation, or lack thereof, of another labeled nucleotide in the primer.

111. The method of claim 110, wherein the labeled nucleotides provided in (b) or (d) are non-terminated.

112. The method of claim 110, wherein the nucleotides provided in (c) are non-terminated.

113. The method of claim 110, wherein the plurality of first flow cycles and the plurality of third flow cycles follow a first flow order, and wherein the plurality of second flow cycles follows a second flow order different from the first flow order.

114. The method of claim 110, wherein the plurality of first flow cycles follows a first flow order, the plurality of second flow cycles follows a second flow order different from the first flow order, and the plurality of third flow cycles follows a third flow order, distinct from the first flow order and second flow order.

115. The method of claim 110, wherein the first sequence comprises a first copy portion of a template nucleic acid molecule and the third sequence comprises the template nucleic acid molecule, and the method further comprises comparing, or combining, first sequencing data corresponding to the one or more signals detected in step (b) and second sequencing data corresponding to the one or more signals detected in step (d), to determine at least a portion of the first sequence.

116. A method for sequencing, comprising:
(a) providing a nucleic acid molecule comprising a first sequence and a second sequence, wherein the first sequence and the second sequence are identical;
(b) sequencing the first sequence by, for each cycle of a plurality of first flow cycles, (i) providing labeled nucleotides of a first combination of three base types to a primer hybridized to the nucleic acid molecule, (ii) detecting one or more signals indicative of incorporation, or lack thereof, of one or more labeled nucleotides of the first combination of three base types in the primer, and (iii) providing nucleotides of a fourth base type different from the three base types in the first combination; and
(c) sequencing the second sequence by, for each cycle of a plurality of second flow cycles, (i) providing labeled nucleotides of a second combination of three base types to the primer, wherein the second combination is different from the first combination, (ii) detecting one or more signals indicative of incorporation, or lack thereof, of one or more labeled nucleotides of the second combination of three base types in the primer, and (iii) providing nucleotides of a fifth base type different from the three base types in the second combination.

117. The method of claim 116, wherein the labeled nucleotides provided in (b) or (c) are non-terminated.

118. The method of claim 116, wherein the labeled nucleotides provided in (b) and (c) are non-terminated.

119. The method of claim 116, wherein nucleotides of the fourth base type provided in step (b) are labeled, and step (b) further comprises (iv) detecting one or more additional signals indicative of incorporation, or lack thereof, of one or more additional labeled nucleotides of the fourth base type.

120. The method of claim 116, wherein nucleotides of the fifth base type provided in step (c) are labeled, and step (c) further comprises (iv) detecting one or more additional signals indicative of incorporation, or lack thereof, of one or more additional labeled nucleotides of the fifth base type.

121. The method of claim 116, further comprising comparing, or combining, first sequencing data corresponding to the one or more signals detected in step (b) and second sequencing data corresponding to the one or more signals detected in step (c), to determine at least a portion of the first sequence.
